(19) United States
(12) Patent Application Publication
(10) Pub. No.: US 2008/0233589 A1
(43) Pub. Date: Sep. 25, 2008

(54) Complementation Systems Utilizing Complexes of Heteroproteins

(75) Inventor: David Piwnica-Worms, Ladue, MO (US)

Correspondence Address: POLSINELLI SHALTON FLANIGAN SUELTHAUS PC, 100 SOUTH FOURTH STREET, SUITE 100, SAINT LOUIS, MO, 63102-1825, US

(73) Assignee: WASHINGTON UNIVERSITY IN ST. LOUIS, St. Louis, MO (US)

(21) Appl. No.: 12/064,825

(22) PCT Filed: Aug. 31, 2006
(86) PCT No.: PCT/US2006/034069

§ 371 Date: Mar. 27, 2008

Related U.S. Application Data
(60) Provisional application No. 60/713,489, filed on Sep. 1, 2005.

(51) Int. Cl. C12Q 1/68; C07H 21/00

(52) U.S. Cl. 435/6; 536/23.1

(57) ABSTRACT

The present invention provides heterologous complementation systems and methods of using the systems to detect molecular interactions. In particular, the heterologous complementation systems comprise polypeptide fragments derived from heterologous polypeptides. If a molecular interaction occurs, then the heterologous polypeptide fragments are able to associate and produce a detectable signal.

### CROSS REFERENCE TO RELATED APPLICATIONS

This application claims priority to PCT International Patent Application No. PCT/US2006/034069 filed on Aug. 31, 2006, which claimed priority to U.S. Provisional Patent Application Ser. No. 60/713,489 filed on Sep. 1, 2005, each of which is hereby incorporated by reference in its entirety.

### GOVERNMENTAL RIGHTS

This invention was made with Government support under grant no. P50 CA94056 awarded by the National Institutes of Health. The Government has certain rights in this invention.

### FIELD OF THE INVENTION

The present invention generally relates to detecting biomolecule interactions. More specifically, the present invention provides constructs for a protein complementation system and methods of using the protein complementation system to detect biomolecule interactions.

### BACKGROUND OF THE INVENTION

Protein complementation was devised recently as a new method to investigate the interaction of biomolecules, particularly proteins. Protein complementation depends on the division of a monomeric protein into two separate fragments that do not spontaneously reassemble and function. Each of the two fragments is attached to a biomolecule. If the two biomolecules interact, the complementary fragments are brought into close proximity and can thereby form a functional protein, capable of producing a detectable signal. One of the first protein complementation assays for reporting purposes utilized β-galactosidase as the reporter. A dihydrofolate reductase (DHFR) system has also been utilized to measure intracellular increases in fluorescence, due to the induction of a protein interaction containing DHFR fragments. While these methods have many advantages over two-hybrid and fluorescence resonance energy transfer (FRET)-based approaches, they are still limited by the need to accumulate product several fold to measure absorption properties, which requires several minutes to hours. Thus, the real time activity of the protein interactions being probed cannot be monitored. This type of temporally-restricted assay is also limited because of the inherent lability of protein interactions and cellular function. Additionally, these particular protein complementation methods are still limited to lysates and cell culture systems.

Previous feasibility studies with Renilla (sea pansy) luciferase monomeric complementation demonstrated the potential to observe protein-protein interactions in living animals. The luciferase fragments, however, suffered from constitutive activity of the N-terminus fragment, and the blue-green emission spectrum of Renilla luciferase penetrated tissues poorly. Furthermore, coelenterazine, the chromophoric substrate for Renilla luciferase, is transported by MDR1 P-glycoprotein, complicating applications of Renilla luciferase in vivo.

A need therefore exists for a protein complementation system that can be used to monitor the real time activity of a protein or other biomolecule. A need also exists for a protein complementation system that can be used to investigate the interaction of proteins or other biomolecules in vivo. In particular, a need exists for a protein complementation system that exhibits a favorable combination of color, output, and fold-induction, for selected applications, allowing for enhanced imaging of interactions between biomolecules in vivo.

### SUMMARY OF THE INVENTION

One aspect of the present invention provides a heterologous complementation system. The heterologous complementation system comprises a first nucleic acid construct and a second nucleic acid construct. The first nucleic acid construct encodes a first biomolecule and a first polypeptide fragment, and the second nucleic acid construct encodes a second biomolecule and a second polypeptide fragment. The first and second polypeptide fragments are each derived from a heterologous polypeptide. Upon expression of the first and second biomolecules and the first and second polypeptide fragments, and if the first biomolecule interacts with the second biomolecule, then the first polypeptide fragment associates with the second polypeptide fragment to produce a detectable signal.

Another aspect of the invention encompasses a method for detecting molecular interactions. The method comprises combining a first nucleic acid construct that encodes a first biomolecule and a first polypeptide fragment with a second nucleic acid construct that encodes a second biomolecule and a second polypeptide fragment. The first polypeptide fragment and the second polypeptide fragment are each derived from a heterologous polypeptide. The contacting occurs under conditions such that the first biomolecule, the first polypeptide fragment, the second biomolecule and the second polypeptide fragment are expressed. The method further comprises determining whether a detectable signal is produced. The detectable signal is produced by association between the first polypeptide fragment and the second polypeptide fragment if the first biomolecule interacts with the second biomolecule.

Other aspects and features of the invention will in part be apparent and in part pointed out hereinafter.

### Reference to Color Figures

This application file contains at least one drawing executed in color. Copies of this patent application publication with color drawings will be provided by the U.S. Patent and Trademark Office upon request and payment of the necessary fee.

### DESCRIPTION OF THE FIGURES

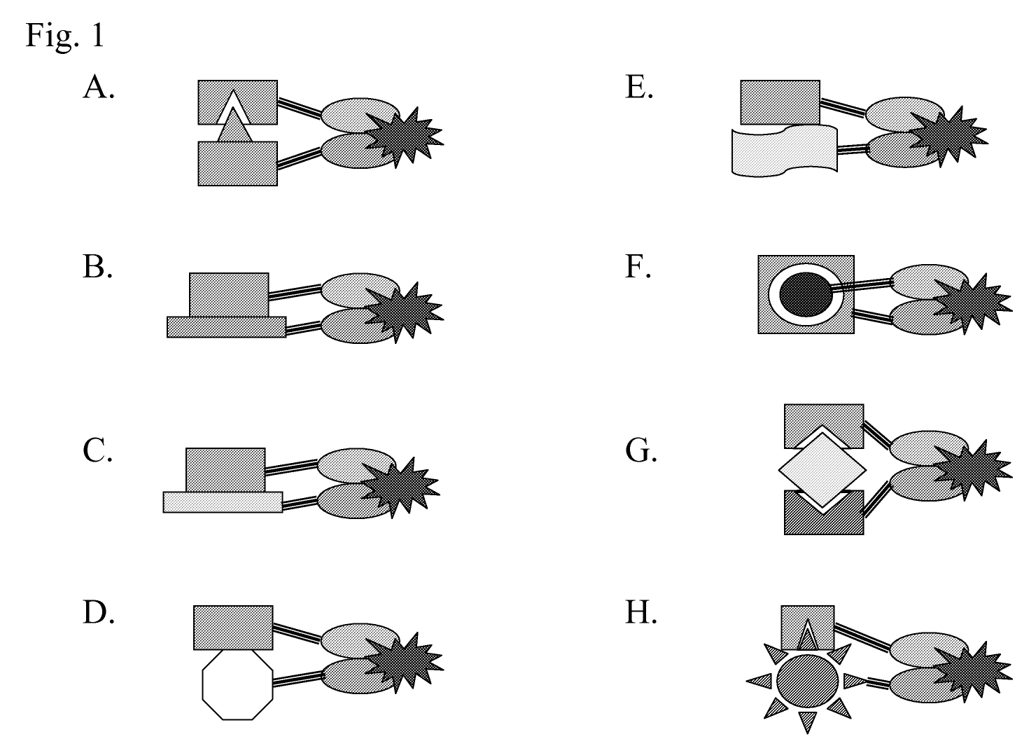

FIG. 1 depicts diagrams of several different embodiments of the invention. Panel A represents an interaction between two constructs of the invention that comprise two distinct proteins (green rectangle shapes). Their interaction leads to the association of the two polypeptide fragments (bright green and salmon ellipses) and the subsequent production of a detectable signal (red starburst). Panel B represents an interaction between two constructs of the invention: one comprises a protein (green rectangle shape) and the other a DNA sequence (pink rectangle). Their interaction leads to the association of the two polypeptide fragments (bright green and salmon ellipses) and the subsequent production of a detectable signal (red starburst). Panel C represents an interaction between two constructs of the invention: one comprises a protein (green rectangle shape) and the other a RNA sequence (lavender rectangle). Their interaction leads to the association of the two polypeptide fragments (bright green and salmon ellipses) and the subsequent production of a detectable signal (red starburst). Panel D represents an interaction between two constructs of the invention: one comprises a protein (green rectangle shape) and the other a carbohydrate (yellow octagon). Their interaction leads to the association of the two polypeptide fragments (bright green and salmon ellipses) and the subsequent production of a detectable signal (red starburst). Panel E represents an interaction between two constructs of the invention: one comprises a protein (green rectangle shape) and the other a lipid (blue wave). Their interaction leads to the association of the two polypeptide fragments (bright green and salmon ellipses) and the subsequent production of a detectable signal (red starburst). Panel F represents an interaction between two constructs of the invention: one comprises a protein (green rectangle shape) and the other a small molecule (dark purple circle). Their interaction leads to the association of the two polypeptide fragments (bright green and salmon ellipses) and the subsequent production of a detectable signal (red starburst). Panel G represents an interaction between two constructs of the invention that comprise two distinct proteins (green rectangle shapes) and an intermediate (pink diamond). The interaction of the intermediate with the two protein constructs leads to the association of the two polypeptide fragments (bright green and salmon ellipses) and the subsequent production of a detectable signal (red starburst). Panel H represents an interaction between two constructs of the invention: one comprises a protein (green rectangle shape) and the other a microbe (brown shape). Their interaction leads to the association of the two polypeptide fragments (bright green and salmon ellipses) and the subsequent production of a detectable signal (red starburst). The black lines represent linkers.

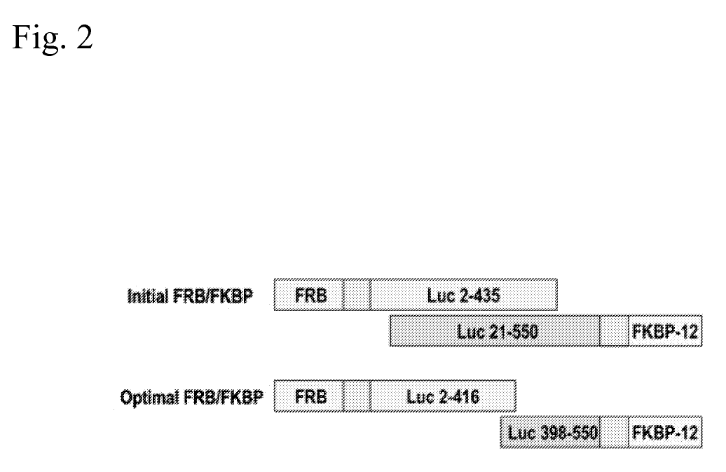

FIG. 2 illustrates the starting firefly luciferase N- and C-terminal fragments, and what the optimal firefly luciferase fragments were after incremental truncation and selection. Luker et al. (2004) Proc. Natl. Acad. Sci. USA 101,12288-12293.

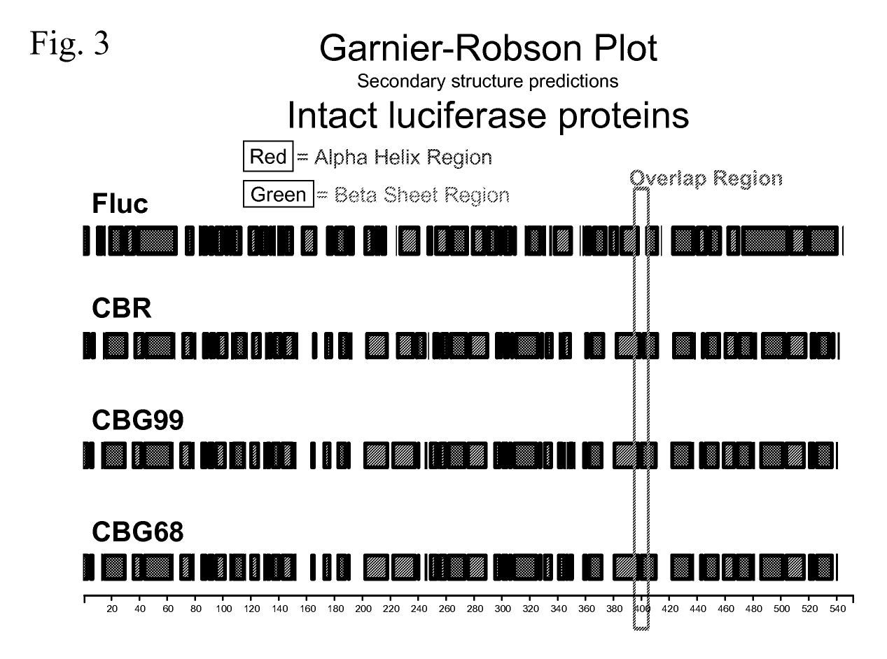

FIG. 3 depicts the high level of secondary structure homology between the different isoforms of luciferases that use D-luciferin as a substrate, despite the modest sequence identity between different luciferase isoforms. The Garnier-Robson plot was performed using PROTEAN. The Overlap Region is located at both the N- and C-termini of the C- and N-terminus luciferase constructs, respectively. FLuc is firefly luciferase; CBR is click beetle red luciferase; CBG99 is click beetle green 99 luciferase; and CBG68 is click beetle green 68 luciferase.

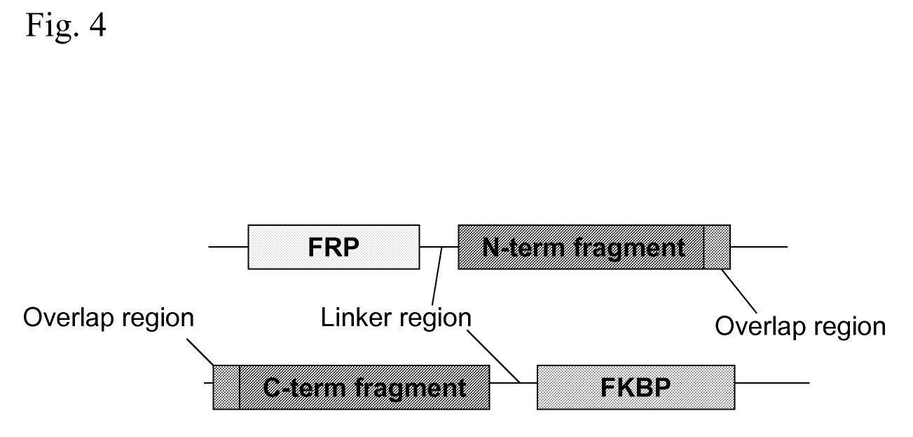

FIG. 4 illustrates the general design of the constructs. The N-terminal fragment and the C-terminal fragment represent N- and C-terminal fragments of the selected heterologous polypeptide fragments. An example of the overlap region is depicted in FIG. 3, and when the polypeptide fragments are derived from firefly luciferase, are represented by amino acids 398-416, and when the polypeptide fragments are derived from click beetle luciferase, are represented by amino acids 394-413.

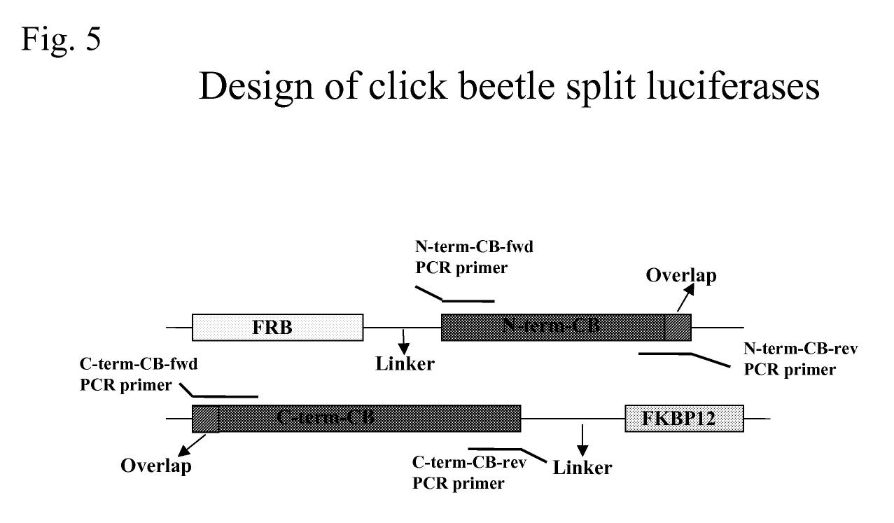

FIG. 5 depicts the design of click beetle split luciferases. Primers to click beetle luciferase red and green were synthesized and the fragments PCR amplified and ligated as illustrated.

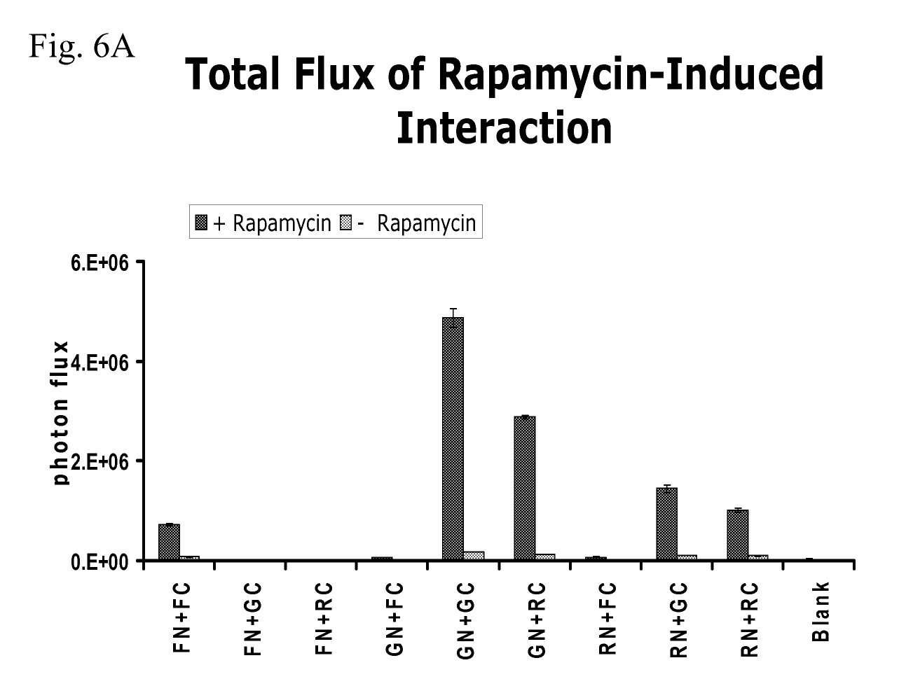

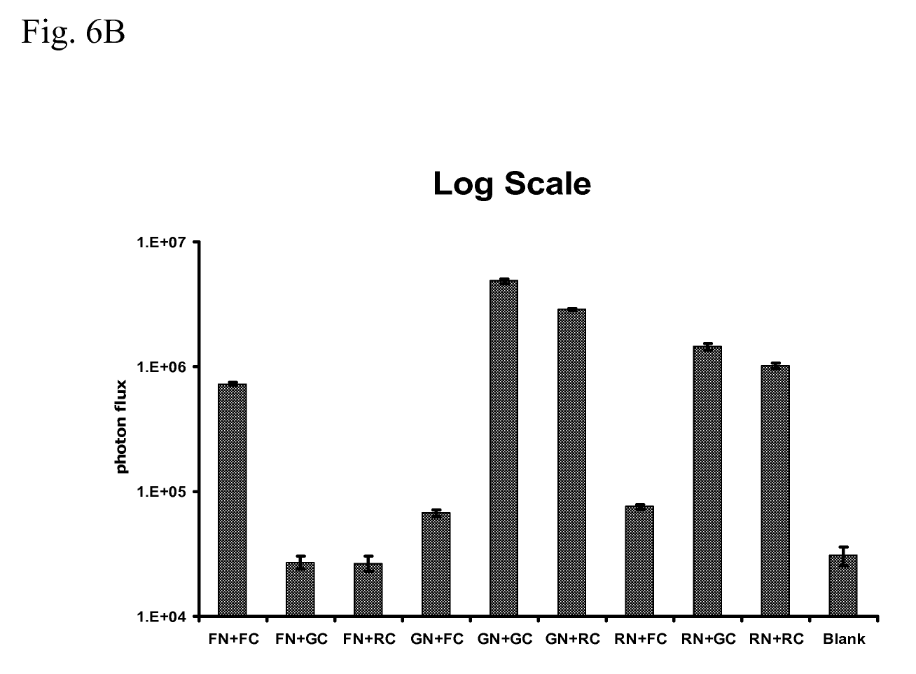

FIG. 6 depicts a graph that represents the total flux of the split luciferase pairs compared to a blank (far right side of graph). (A) The red bars represent cells exposed to 100 nM rapamycin for 8 hrs at 37 degrees C., while the blue bars represent cells in the absence of rapamycin. Bioluminescence images taken for 60 sec with bin 8. (B) The same data as in (A) except the y-axis is a log scale. F=firefly luciferase, R=red click beetle luciferase, G =green click beetle 99 luciferase, N=N-terminus construct with FRP, C=C-terminus construct with FKBP. Photon flux units=photons/sec.

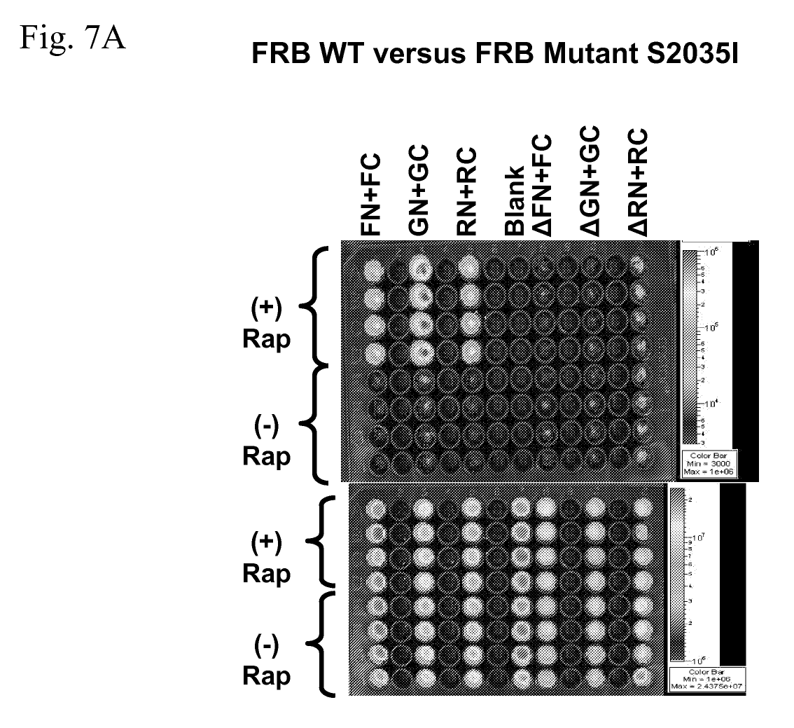

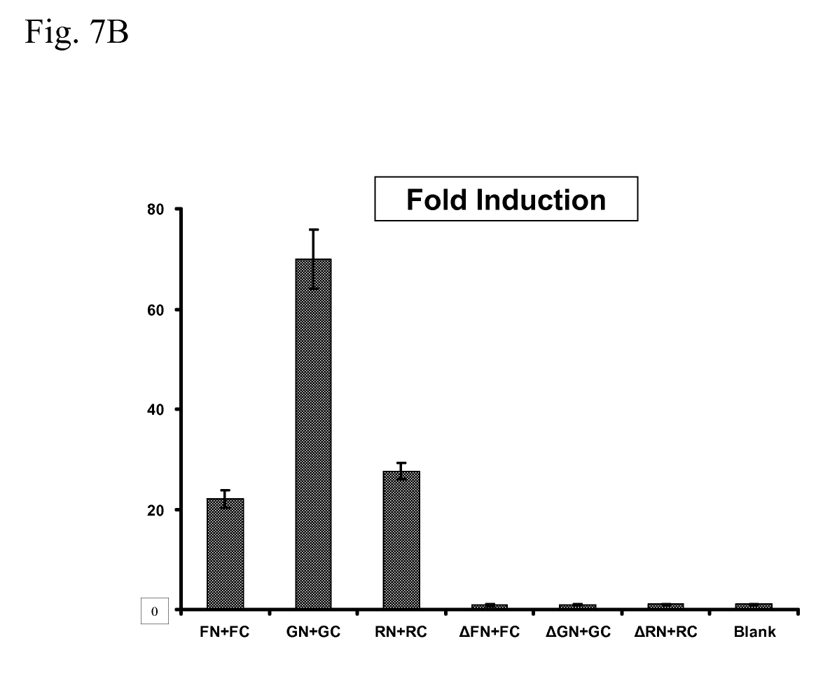

FIG. 7 illustrates the fold induction of the native split luciferase pairs fused to wild type and mutant S20351 FRB upon addition of rapamycin. FRB mutant S20351 (ΔF) blocks rapamycin-induced binding of FRB to FKBP. (A) Photographic images of HEK293T cells transfected with the constructs. The ΔF mutants show no induction. Renilla is used as a construct transfection control. (B) Graph depicting fold induction. Fold induction is calculated as the signal ratio between the cultures exposed to rapamycin and those not exposed. Abbreviations as in FIG. 6.

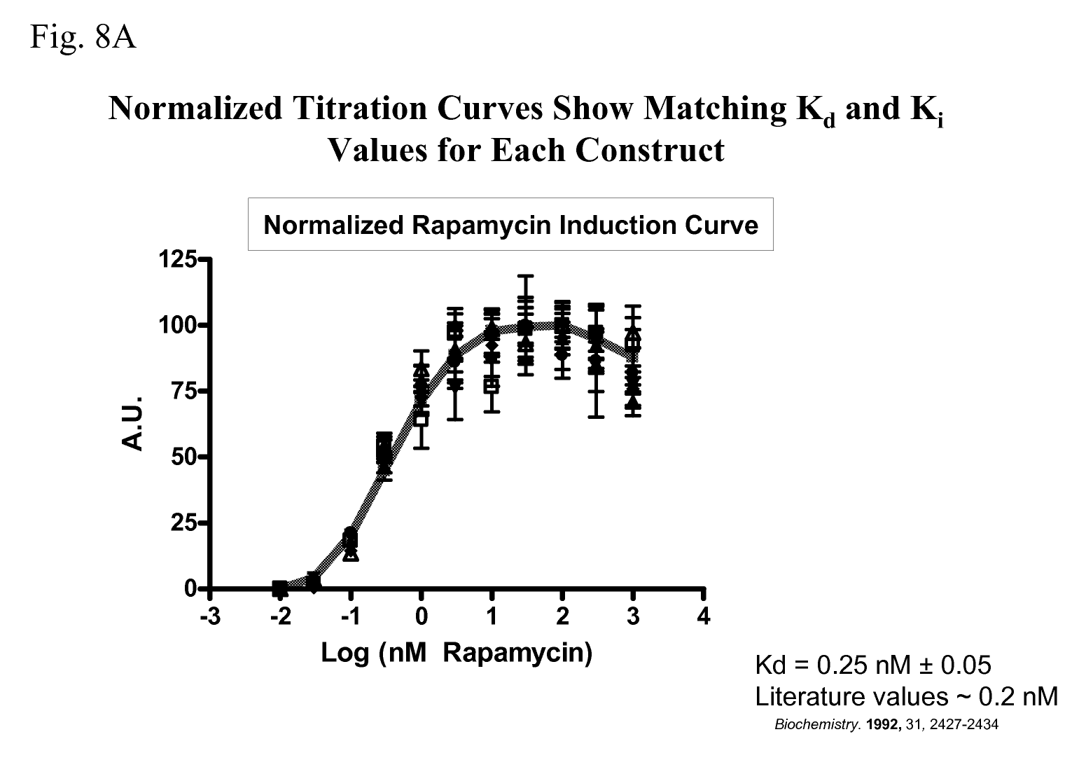

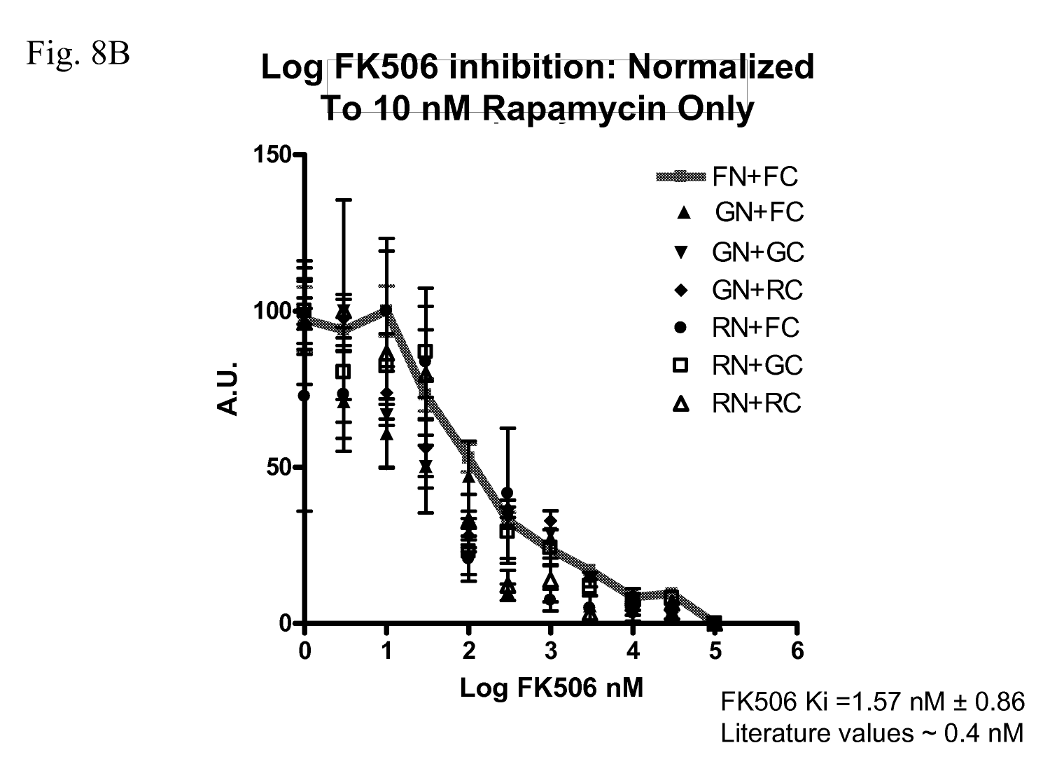

FIG. 8: The graph represents the rapamycin-induced protein interaction titration curve for all polypeptide fragment pairs. 48 hours post transfection, HEK293T cells were treated for 8 hours with (A) rapamycin at various concentrations, or (B) increasing amounts of FK506 (an inhibitor of FKBP/FRB interaction) and 10 nM rapamycin . Red line shows Firefly luciferase split. No significant shift in Kd or Ki was observed regardless of color, total output or fold induction for the various hetero-protein pairs. Abbreviations as in FIG. 6.

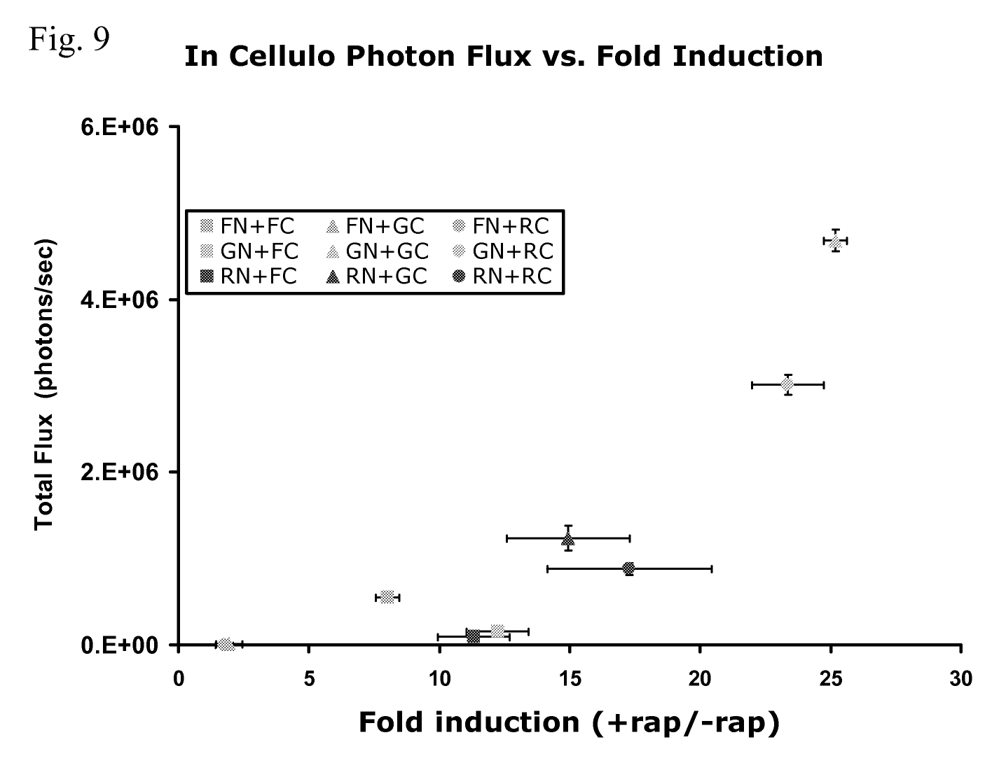

FIG. 9 depicts a graph that represents a comparison between fold induction (calculated as described in FIG. 7) and photon flux of the various split luciferase pairs in live cells. Note, the optimal luciferase pairs are GN:GC, GN:RC, RN:RC and RN:GC. Abbreviations as in FIG. 6.

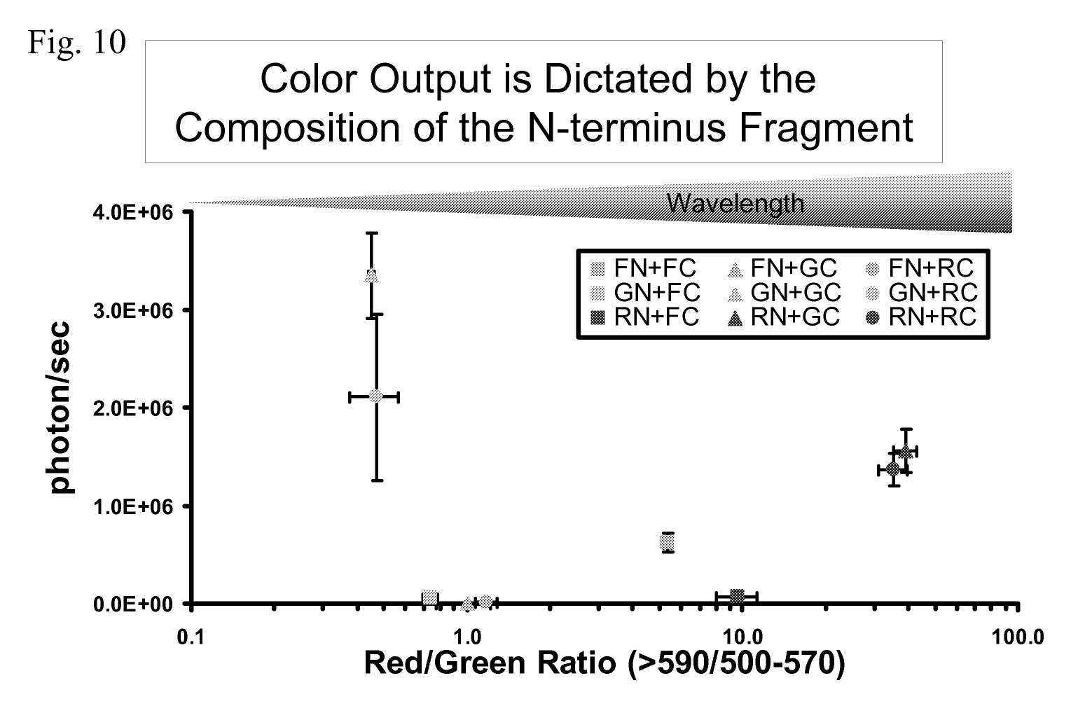

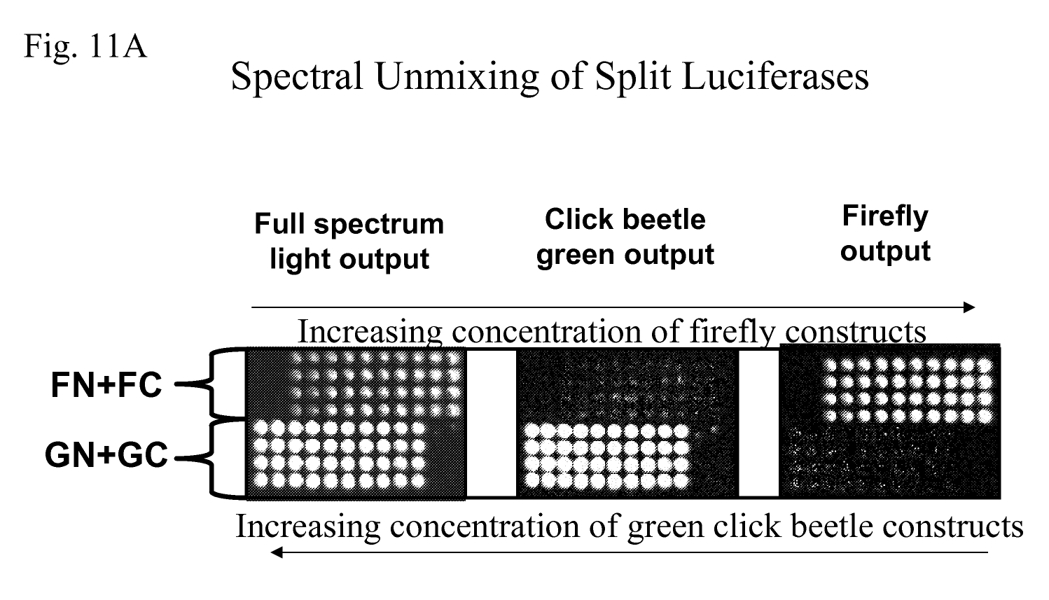

FIG. 10 shows that the detected color tracks with the N-terminus of the various hetero-protein pairs. The graph represents the color produced by the split luciferase pairs in comparison to the initial split firefly construct. HEK293T cells were transfected for 48 hours and then incubated for 8 hours with 100 nM rapamycin. Red and green emission was detected with a 590 nm long-pass filter and 500-570 nm band-pass filter, respectively. Relative color is determined by measuring the 60 sec image photon output values in each filter and calculating the ratio of red emission to green emission. Abbreviations as in FIG. 6.

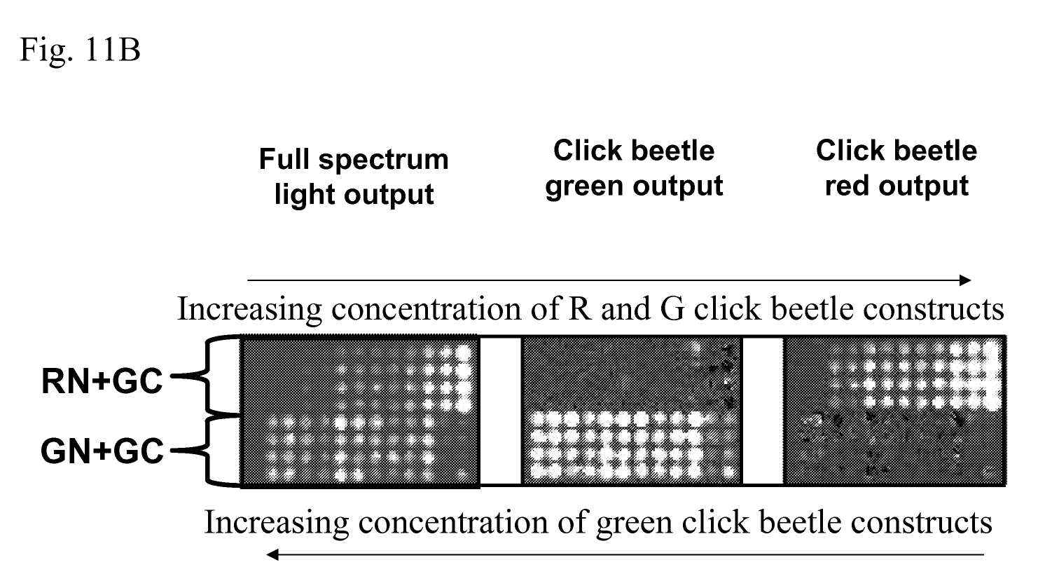

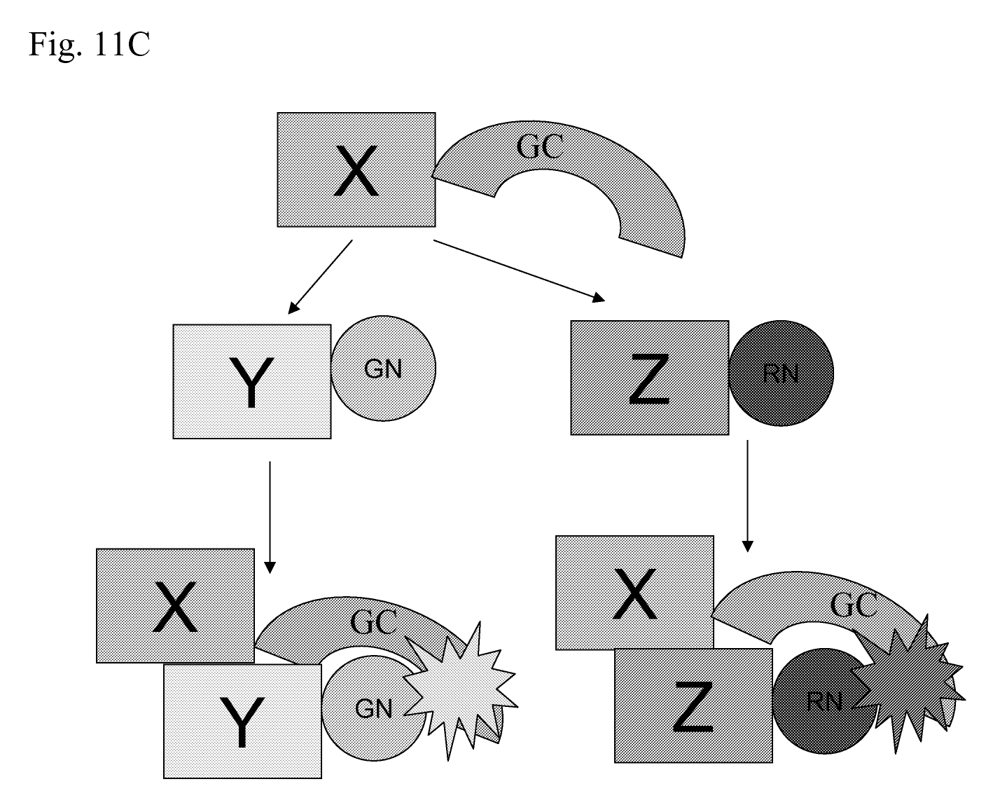

FIG. 11 depicts photographs and illustrations showing spectral unmixing in different live cells. Various ratios of DNA from the indicated polypeptide fragment pairs were transfected into HEK293T cells and imaged with red (>590 nm) and green (500-570 nm) filters to separate the colors as shown in (A) and (B). Using a previously published bioluminescence unmixing algorithm (Gammon et al. 2006, Anal Chem 78:1520-1527), different luciferase colored-tagged protein interactions were quantified simultaneously. Thus, the location and type of various colored luciferase-tagged protein interactions (e.g., FN+FC and GN+GC in (A) and RN+GC and GN+GC in (B)) could be identified on the 96-well plates. (C) illustrates the reaction in (B). If X biomolecule interacts with Y biomolecule, GN and GC will associate, producing a green signal. If X biomolecule interacts with Z biomolecule, RN and GC will associate, producing a red signal. Abbreviations as in FIG. 6.

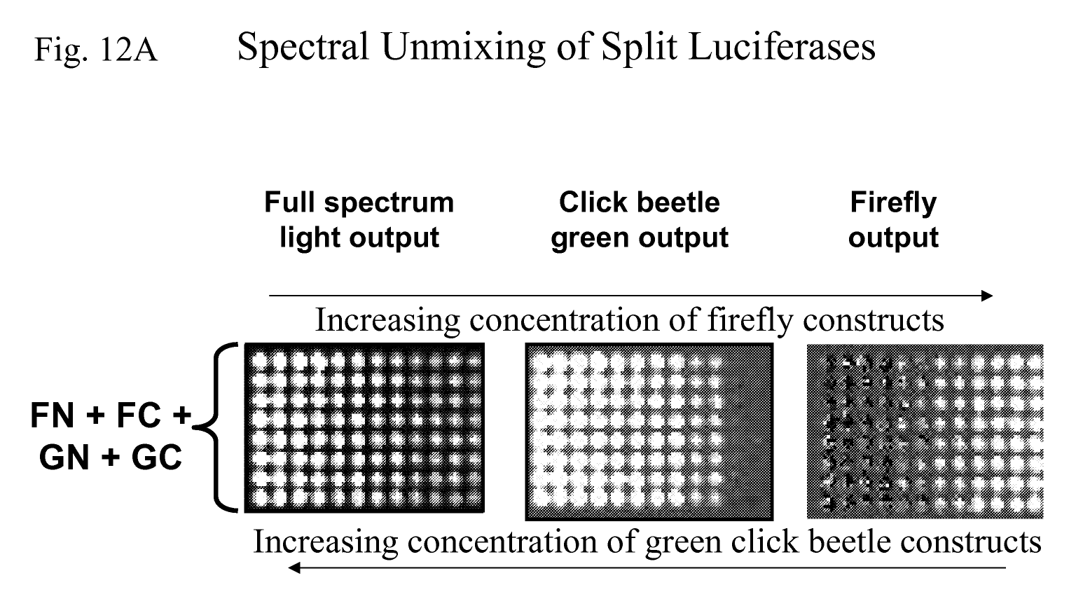

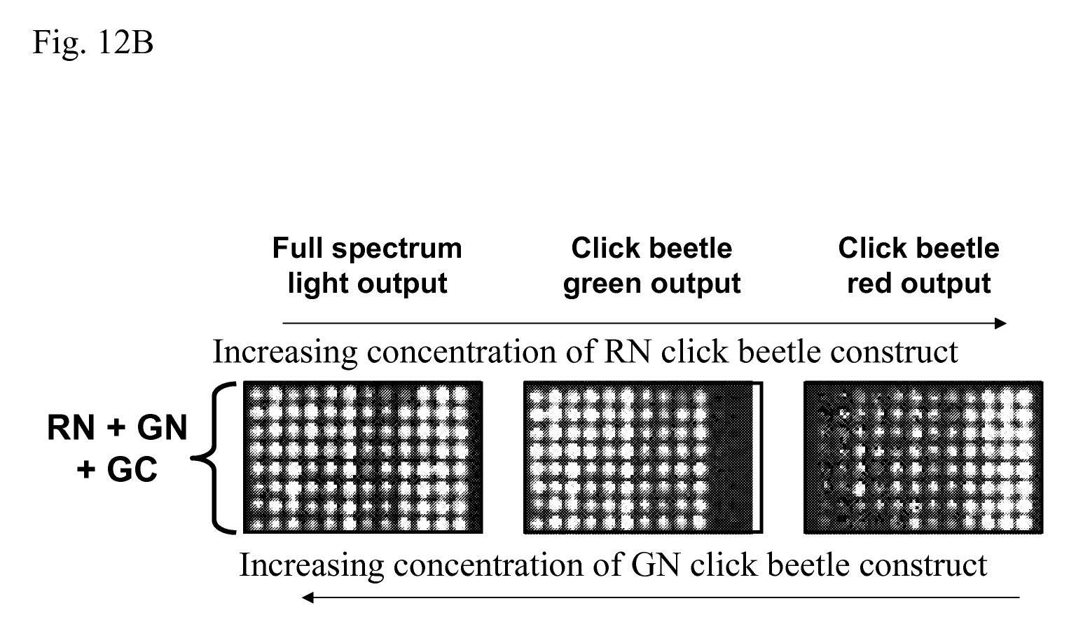

FIG. 12 depicts photographs showing spectral unmixing in the same live cells. Varying DNA concentrations were co-transfected into the same cells. Herein, two polypeptide fragment pairs could be simultaneously imaged and deconvoluted in the same cells (e.g., FN+FC and GN+GC) with two different colored filters (A) or importantly, two different N-terminal derived polypeptide fragments could be simultaneously imaged and interactions with the same C-terminal derived polypeptide fragment deconvoluted (e.g., RN+GN+GC) in the same cells (B). The latter example provides a novel tool for interrogation of branch points in protein-protein interaction pathways and molecular switches in living cells in real time. Abbreviations as in FIG. 6.

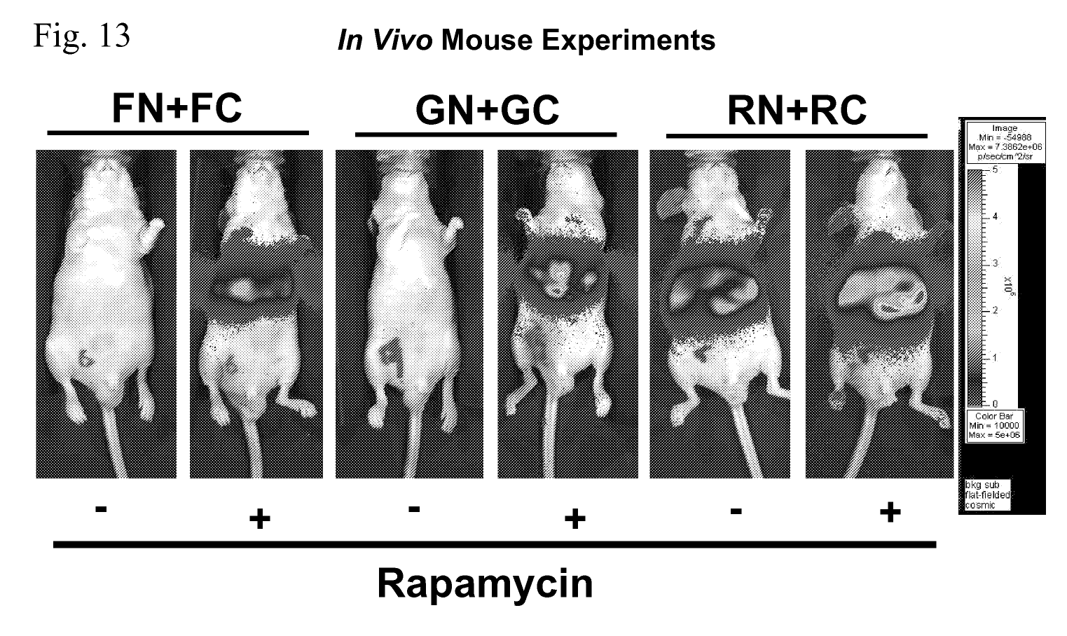

FIG. 13 depicts images of representative mice imaged pre and post rapamycin to illustrate the use of different polypeptide fragment pairs in live animals. Hepatocellular somatic gene transfer was performed by hydrodynamic injections of 15 mg total DNA (various luciferase polypeptide fragment pairs plus Renilla luciferase) per mouse. After 18 hours, mice were injected with 150 mg/kg D-Luciferin IP and imaged with an IVIS 100 CCD camera to establish baseline photon output. Mice were then treated with rapamycin (IP: 1 mg/kg) and re-imaged 6 hours post-rapamycin to visualize induced protein complementation in vivo. Abbreviations as in FIG. 6.

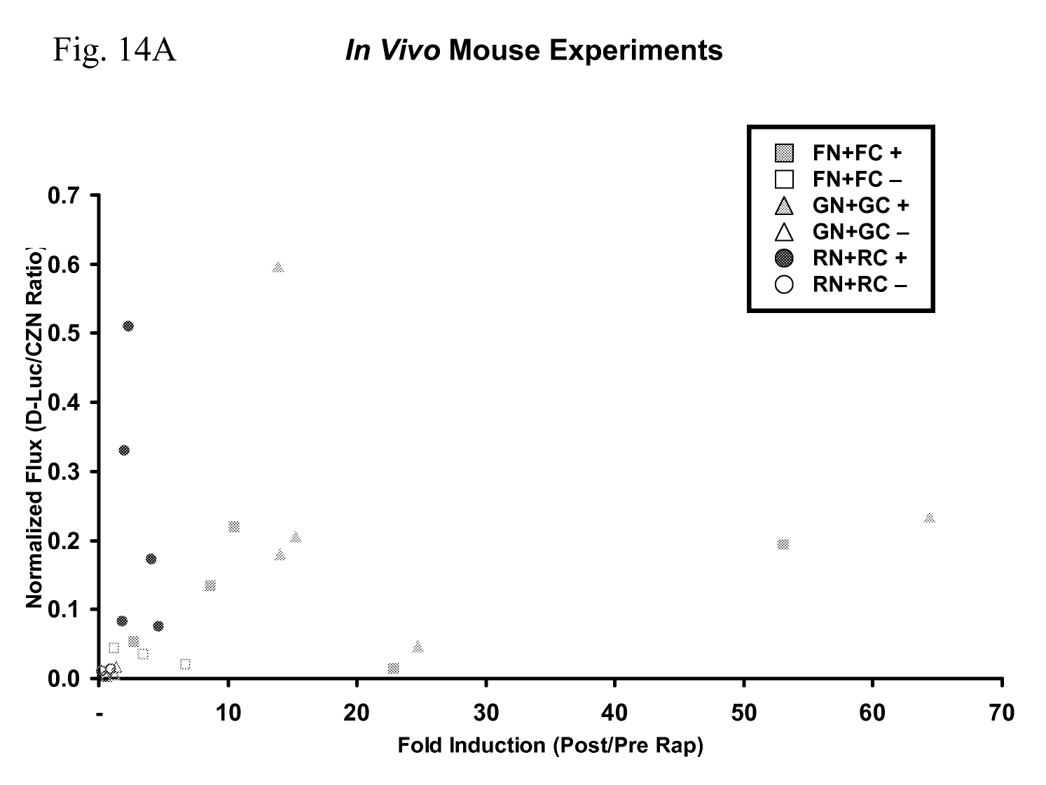

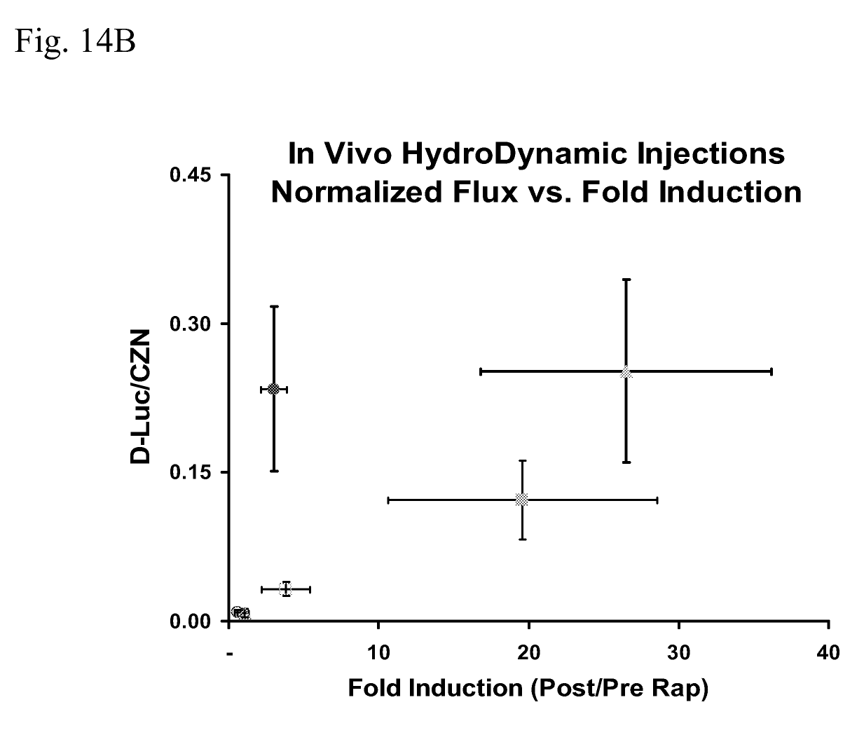

FIG. 14 illustrates the quantification of photon output in vivo. The graph in (A) represents a comparison between fold induction (calculated as described in FIG. 7) and normalized photon flux (D-luciferin/coelenterazine (CZN) ratio) of the various split luciferase pairs in individual living animals. In the mice illustrated in FIG. 13, coelenterazine (IV: 1 mg/kg) was also injected as described previously (Pichler et al., 2005, Clin Cancer Res 11:4487-4494) to measure Renilla luciferase activity for monitoring transfection efficiency. D-Luciferin photon outputs (polypeptide fragment pairs) were normalized to coelenterazine photon output (Renilla luciferase) and is presented as a ratio of Split Luc/Renilla. Pre-treatment (−), Post-rapamycin (+). The graph in (B) shows the average +/− SEM of all mouse data. Abbreviations as in FIG. 6.

### DETAILED DESCRIPTION OF THE INVENTION

Advantageously, the protein complementation system of the present invention allows for real time enhanced imaging of biomolecule interactions both in vitro and in vivo. The system comprises two polypeptide fragments, each of which is derived from a heterologous polypeptide. The protein complementation system may be used to investigate interactions between biomolecules, including proteins, lipids, and carbohydrates. Each polypeptide fragment is attached to a biomolecule, and together, the two fragments function as a reporter. If the two biomolecules interact, the two polypeptide fragments come together to form a functional protein and produce a detectable signal, e.g., an optical signal. For selected applications, the detectable signal may be inducible. Advantageously, the combinations of heterologous polypeptide fragments utilized in the complementation systems of the present invention exhibit a favorable combination of color, output, and fold-induction, whether used in vitro or in vivo.

### I. Heterologous Complementation System

One aspect of the invention provides a heterologous complementation system comprising at least one pair of nucleic acid constructs. Irrespective of the embodiment, the constructs will encode combinations of heterologous polypeptide fragments that are utilized to identify the interaction between biomolecules in various assays of the invention. Each combination of heterologous polypeptide fragments will include a first polypeptide fragment derived from an N-terminal region of a polypeptide and a second polypeptide fragment derived from a C-terminal region of another polypeptide.

In one embodiment, the heterologous complementation system comprises a first nucleic acid construct and a second nucleic acid construct. The first nucleic acid construct has a nucleotide sequence encoding a first biomolecule and a nucleotide sequence encoding a first polypeptide fragment. The first polypeptide fragment is derived from an N-terminal region of a polypeptide. The second nucleic acid construct has a nucleotide sequence encoding a second biomolecule and a nucleotide sequence encoding a second polypeptide fragment. The second polypeptide fragment is derived from a C-terminal region of another polypeptide.

The polypeptide fragments encoded by each pair of nucleic acid constructs utilized, irrespective of the embodiment, are derived from heterologous polypeptides. According to the methods of the invention, each polypeptide fragment is a portion of a protein that may be utilized as a reporter when associated with another polypeptide fragment, i.e. the first polypeptide fragment may be utilized as a reporter when associated with the second polypeptide fragment. If the biomolecule encoded by the first nucleic acid construct interacts with the biomolecule encoded by the second nucleic acid construct, the two polypeptide fragments come together to form a functional protein and produce a detectable signal.

As will be appreciated by the skilled artisan, several nucleic acid constructs are suitable for use in the present invention. In one embodiment, the first and the second nucleic acid constructs together have formula (I):

R1-R2-R3-R4; and

R5-R6-R7-R8; (I)

wherein:

R1 is a nucleotide sequence encoding a first biomolecule;

R2 is a nucleotide sequence encoding a flexible linker;

R3 is a nucleotide sequence encoding a first polypeptide fragment derived from an N-terminal region of a polypeptide;

R4 and R5 are nucleotide sequences encoding overlap regions;

R6 is a nucleotide sequence encoding a second polypeptide derived from a C-terminal region of a polypeptide;

R7 is a nucleotide sequence encoding a flexible linker; and

R8 is a nucleotide sequence encoding a second biomolecule.

In an alternative embodiment, the first nucleic acid construct and the second nucleic acid construct together have formula (II),

R1-R3; and

R6-R8; (II)

wherein:

R1, R3, R6, and R8 are as described for constructs having formula (I).

In this embodiment the nucleotide sequence encoding the first biomolecule is fused in frame with the nucleotide sequence encoding the first polypeptide fragment and the nucleotide sequence encoding the second polypeptide fragment is fused in frame with the nucleotide sequence encoding the second biomolecule.

In a further embodiment, the first nucleic acid construct and the second nucleic acid construct together have formula (III),

R1-R2-R3; and

R6-R7-R8; (III)

wherein:

R1, R2, R3, R6, R7 and R8 are as described for constructs having formula (I).

In a particularly preferred alternative of this embodiment, R1 encodes FRP; R2 and R7 each encode flexible linkers comprising glycine and/or serine; R3 encodes a polypeptide fragment derived from the N-terminal of a click beetle luciferase; R6 encodes a polypeptide fragment derived from the C-terminal of another click beetle luciferase; and R8 encodes FKBP.

In still an additional embodiment, the first nucleic acid construct and the second nucleic acid construct together have formula (IV),

R1-R3-R4; and

R5-R6-R8; (IV)

wherein:

R1, R3, R4, R5, R6 and R8 are as described for constructs having formula (I).

In a further embodiment for each of the construct pairs having any of formulas I-IV, R1 maybe a nucleotide sequence encoding the first polypeptide fragment, R3 maybe a nucleotide sequence encoding the first biomolecule, R6 maybe a nucleotide sequence encoding the second biomolecule, and R8 might be a nucleotide sequence encoding the second polypeptide fragment.

In still another embodiment, the first nucleic acid construct and the second nucleic acid construct may be linked and form one molecule. Typically, upon expression of the construct, the first biomolecule and first polypeptide fragment will form a polypeptide fused to the second polypeptide fragment and second biomolecule. For example, the fused construct may have formula (V), formula (VI), formula (VII), or formula (VIII),

R1-R2-R3-R4-R2-R5-R6-R2-R8; (V)

R1-R3-R2-R6-R8; (VI)

R1-R2-R3-R2-R6-R2-R8; (VI)

R1-R3-R4-R2-R5-R6-R8; (VII)

wherein:

R1, R2, R3, R4, R5, R6, and R8 are as described for constructs having formula (I).

a. Heterologous Polypeptide Fragments

A variety of first and second polypeptide fragments (i.e., which are encoded by R3 and R6, respectively) are suitable for use in the present invention. Generally speaking, the first polypeptide fragment and second polypeptide fragment together form a pair of heterologous protein fragments, that when brought into close proximity, reconstitute to form a functional protein that produces a detectable signal. In this context, the reconstituted protein has reporter activity. The ability to select among a wide range of heterologous polypeptide fragments to fulfill this reporter function allows flexibility in automation, detection mode, instrumentation, cell type, experimental protocol, sensitivity, specificity, and cost. Methods for selecting polypeptide fragment combinations suitable for use in the invention are described below. Generally, when selecting polypeptides, one skilled in the art may consider one or more of the following factors: the size and nature of the polypeptide, typically relatively small and monomeric; structural and functional information available about the protein; the availability of systems for the activity of the protein (in vivo and in vitro); and whether or not overexpression of the polypeptide in eukaryotic and prokaryotic cells has been demonstrated. In preferred embodiments, pairs of suitable polypeptide fragments will typically be from the same protein class, will utilize the same substrate, or will have a similar mechanism of action.

Non-limiting examples of suitable proteins from which the first polypeptide fragment and second polypeptide fragment may be selected include dihydrofolate reductase (DHFR), fluorescent proteins (e.g. GFP, RFP, CFP, YFP, etc.), luciferases, aminoglycoside kinase (AK), thymidine kinase, hygromycin-B-phosphotransferase, adenosine deaminase, L-histidinol:NAD⁺ oxidoreductase, xanthine-guanine phosphoribosyl transferase (XPRT), glutamine synthetase, asparagine synthetase, puromycin N-acetyltransferase, aminoglycoside phosphotransferase, bleomycin binding protein, cytosine methyltransferase, O6-alkylguanine alkyltransferase, glycinamide ribonucleotide (GAR) transformylase, glycinamide ribonucleotide synthetase, phosphoribosyl-aminoimidazole synthetase, formylglycinamide ribotide amidotransferase, phosphoribosyl-aminoimidazole carboxamide formyltransferase, fatty acid synthease, IMP dehydrogenase, DT-diaphorase, NADH-diaphorase, glutathione-S-transferase, chloramphenicol acetyltransferase, uricase, secreted alkaline phosphatase (SEAP), B-glucuronidase, and tyrosinase.

Preferably, the first polypeptide fragment and the second polypeptide fragment are each derived from an enzyme. In certain embodiments, the first and second polypeptide fragments are independently selected from the group consisting of a kinase, phosphatase, protease, exopeptidase, endopeptidase, extracellular metalloprotease, lysosomal protease, HIV protease, transferase, synthase, carboxylase, hydrolase, isomerase, ligase, oxidoreductase, esterase, alkylase, glycosidase, phospholipase, endonuclease, ribonuclease, and a beta-lactamase.

In certain embodiments, the first and second polypeptide fragments are each derived from a fluorescent protein. Non-limiting examples of fluorescent proteins include GFP, EGFP, Emerald, Azami Green, Monomeric Azami Green, CopGFP, AceGFP, ZsGreen1, EBFP, GFPuv, Sapphire, T-sapphire, ECFP, cerulean, AmCyan1, Midoriishi-Cyan, YFP, EYFP, Citrine, Venus, PhiYFP, ZsYellow1, Kusabira-Orange, Monomeric Kusabira-Orange, mOrange, tdimer2(12), mRFP1, DsRed-Express, DsRed2, DsRed-Monomer, HcRed-Tandem, HcRed1, AsRed2, eqFP611, mPlum, mRasberry, mCherry, mStrawberry, mTangerine, tdTomato, and Jred.

In a preferred embodiment, each of the polypeptide fragments is derived from a luciferase. Generically speaking, a luciferase is an enzyme that catalyzes a light producing chemical reaction. Different classes of luciferases may use different substrates to produce light. In certain embodiments of the invention, the polypeptide fragments are derived from heterologous luciferases that utilize D-luciferin as a substrate. In certain preferred embodiments of the invention, the heterologous polypeptide fragments of a luciferase are selected from the group consisting of firefly luciferases and click beetle luciferases. Firefly luciferases include enzymes derived from any one of some 1100 species of firefly. Click beetle luciferases include enzymes derived from any one of some 7000 species of click beetle. In certain particularly preferred embodiments, the first polypeptide fragment is derived from a first click beetle luciferase and the second polypeptide fragment is derived from a second click beetle luciferase.

Preferred examples of heterologous luciferase polypeptides include polypeptides from firefly luciferase, click green beetle luciferase, and click red beetle luciferase. In one embodiment, the first polypeptide fragment is derived from a first click beetle green luciferase, and the second polypeptide fragment is derived from a second click beetle green luciferase. Non-limiting examples of click beetle green luciferases include green68 and green99. In another embodiment, the first polypeptide fragment is derived from a click beetle green luciferase, and the second polypeptide fragment is derived from a click beetle red luciferase. In yet another embodiment, the first polypeptide fragment is from a click beetle red luciferase, and the second polypeptide fragment is from a click beetle green luciferase. In still yet another embodiment, the first polypeptide fragment is from a click beetle red luciferase and the second polypeptide fragment is from a click beetle red luciferase. Alternatively, in one embodiment, the first polypeptide fragment is derived from an N-terminal region of a click beetle green luciferase, and the second polypeptide fragment is derived from a C-terminal region of another luciferase. In another alternative, the first polypeptide fragment is derived from an N-terminal region of a click beetle red luciferase, and the second polypeptide fragment is derived from a C-terminal region of another luciferase.

In some embodiments, there may be a region of overlap (i.e., which are encoded by R4 and R5) between the first polypeptide fragment and the second polypeptide fragment. Typically, the C-terminal end of the first polypeptide fragment overlaps with the N-terminal end of the second polypeptide fragment. In general, the region of overlap between the two fragments will have a similar secondary structure. For example, the region of overlap may have an alpha helical structure. The region of overlap may comprise about 4 amino acid residues, about 8 amino acid residues, about 12 amino acid residues, about 16 amino acid residues, about 20 amino acid residues, about 24 amino acid residues, or about 30 amino acid residues. In embodiments in which the polypeptide fragments are derived from luciferases, the region of overlap may comprise from about 16 amino acid residues to about 22 amino acid residues, or more preferably about 19 amino acid residues. In particular, the region of overlap may comprise a region from amino acid residue 395 to amino acid residue 416 of each click beetle or firefly luciferase.

b. Biomolecules

The first biomolecule and second biomolecule (i.e., which are encoded by R1 and R8, respectively) may include any pair of biomolecules, that when contacted, putatively interact. For example, a biomolecule may be a protein, a peptide, an enzyme, a receptor, an antibody, a small molecule, a ligand, an antigen, a nucleic acid, a lipid, a carbohydrate, or a microbe. FIG. 1 presents non-limiting examples suitable of biomolecules that interact with each other. Interacting biomolecules may include two proteins, a protein and a protein binding factor (e.g., a second protein or peptide, a nucleic acid, a carbohydrate, a lipid, a small molecule, etc), an enzyme and a substrate (e.g., a small molecule, a peptide, a lipid, etc.), a receptor and a ligand (e.g., a hormone, a peptide, a small molecule, etc.), an antibody and an antigen, two proteins and an intermediate protein, or a protein and a microbe.

Proteins may include soluble, filamentous, or membrane-associated proteins. More specifically, proteins may include enzymes, antibodies, transmembrane transport proteins, receptors, ligands, cytoskeletal proteins, hemoproteins, glycoproteins, cell adhesion proteins, protein hormones and growth factors, transcription regulation proteins, and nutrient storage and transport proteins. Nucleic acids may include deoxyribonucleic acid (DNA) and ribonucleic acid (RNA). More specifically, nucleic acids may include complementary DNA (cDNA), single-stranded DNA (ssDNA), double-stranded DNA (dsDNA), small interfering RNA (siRNA), ribozymes, short hairpin RNA (shRNA), ribosomal RNA (rRNA), microRNA, messenger RNA (mRNA), and transfer RNA (tRNA). Lipids may include fatty acids, phospholipids, sphingolipids, glycolipids, and terpenoids (i.e., steroids).

In a preferred embodiment, the first biomolecule may be FRB (i.e., the rapamycin binding fragment of mTOR) and the second biomolecule may be FKBP (i.e., FK506-binding protein-12). In this embodiment, therefore, interaction between the two biomolecules may be induced (i.e., via rapamycin).

c. Optional Linkers

The linkers (i.e., which are encoded by R2 and R5), if present, typically functions to impart flexibility to the construct so as to reduce steric constraints as between the biomolecule and polypeptide fragment. In certain embodiments, the linker may comprise from about 1 to about 20 glycine or serine amino acid residues. In a preferred embodiment, the linker comprises from about 5 to about 10 glycine or serine amino acid residues. In other embodiments, the linker may also include one or more multiple cloning sites.

d. Detectable Signals

Association of the first polypeptide fragment with the second polypeptide fragment forms a functional polypeptide that generates a detectable signal. The detectable signal may be an optical signal, a radioisotopic radioactive emission signal, or a survival signal. In preferred embodiments, the detectable signal is an optical signal. Suitable optical signals include signals such as a fluorescent signal, a luminescent signal, a phosphorescent signal, or a colorimetric signal.

In certain embodiments, the detectable signal may be produced upon cleavage of a substrate by an enzyme (i.e., the associated polypeptide fragments) to produce a colored, fluorescent, luminescent, or phosphorescent product. For example, luciferase, a protein commonly used in nature for bioluminescence (the emission of visible light in living organisms), catalyzes the oxidation of luciferin and thereby produces light. A variety of organisms produce light using a luciferase, including various species of firefly and click beetle, certain fungi, such as Omphalotus olearius (the Jack-O-Lantern mushroom), certain bacteria, such as Vibrio fischeri, and many marine creatures, including Renilla (sea pansy), certain species of squid, and certain species of fish. Some organisms, notably click beetles, have several different luciferase enzymes, i.e., click beetle red luciferase, click beetle green luciferase, each of which can produce different colors from the same luciferin substrate. And, while the particular mechanism of bioluminescence varies from organism to organism, each such mechanism involves luciferase and a substrate, such as luciferin or coelenterazine. Thus, there are many variants of luciferase that the systems of the invention may employ.

In a further embodiment, the complementation systems of the invention may employ an enzyme that interacts with its substrate to produce a fluorescent product. For example, a red fluorescence signal may be generated upon the interaction of two proteins simply by using a fluorophore such as Texas Red-methotrexate in conjunction with the enzyme dihydrofolate reductase (DHFR). A wide spectrum of fluorescence detection systems may be constructed, for example by using any of the BODIPY, Cy3, Cy5, rhodamine, fluorescein, coumarin, or other dyes conjugated to the appropriate substrate. In an iteration of this embodiment, the detectable signal may be based on fluorescence resonance energy transfer (FRET) between donor and acceptor fluorophores linked to a substrate.

In still another embodiment, the association of the polypeptide fragments may produce a protein that is able to generate a fluorescent signal upon exposure to a particular wavelength of light. Examples of suitable fluorescent proteins include GFP, EGFP, Emerald, Azami Green, Monomeric Azami Green, CopGFP, AceGFP, ZsGreen1, EBFP, GFPuv, Sapphire, T-sapphire, ECFP, cerulean, AmCyan1, Midoriishi-Cyan, YFP, EYFP, Citrine, Venus, PhiYFP, ZsYellow1, Kusabira-Orange, Monomeric Kusabira-Orange, mOrange, tdimer2(12), mRFP1, DsRed-Express, DsRed2, DsRed-Monomer, HcRed-Tandem, HcRed1, AsRed2, eqFP611, mPlum, mRasberry, mCherry, mStrawberry, mTangerine, tdTomato, and Jred.

In a further embodiment, the signal may be detected colorimetrically. For example, an enzyme may convert a colorless substrate into a colored reaction product, which than can be detected. In another iteration, an enzyme may convert a soluble substrate into an insoluble, colored reaction product. In a further embodiment, the activity of an enzyme may change a substrate that is one color into a product that is another color. For example, hydrolysis of the amide bond in the beta-lactam ring of the yellow colored substrate nitrocefin by beta-lactamase generates a red colored reaction product.

### II. Methods for Detecting Molecular Interactions

Another aspect of the invention encompasses methods for detecting molecular interactions. In general, the method comprises combining a first nucleic acid construct having a nucleotide sequence encoding a first biomolecule and a nucleotide sequence encoding a first polypeptide fragment with a second nucleic acid construct having a nucleotide sequence encoding a second biomolecule and a nucleotide sequence encoding a second polypeptide fragment. The first and second polypeptide fragments each derived from a heterologous polypeptide. The contacting occurs under conditions that allow expression of the first biomolecule, the first polypeptide fragment, the second biomolecule and the second polypeptide fragment. The method further comprises determining whether a detectable signal is produced. The detectable signal is produced by association of the first polypeptide fragment with the second polypeptide fragment if the first biomolecule and the second biomolecule interact.

The interactions between biomolecules contemplated by the invention include protein-protein interactions, protein-DNA interactions, protein-RNA interactions, protein-lipid interactions, protein-carbohydrate interactions, protein-small molecule interactions, protein-intermediate-protein interactions, and protein-microbe interactions. These interactions are shown in FIG. 1. Biomolecule interaction assays may be constructed to enable visualization, quantitation, and localization of specific biomolecule-biomolecule complexes.

Non-limiting examples of protein-protein interactions may include cell surface receptor-ligand interactions, such as the interaction between a chemokine receptor and a chemokine, membrane protein-secondary messenger interactions, and antibody-ligand interactions. Non-limiting examples of protein-DNA interactions may include DNA binding protein-DNA interactions, such as the interaction between chromatin-binding protein and DNA, as well as transcription factor-DNA interactions. Non-limiting examples of protein-RNA interactions may include ribosomal protein-RNA interactions and interactions between RNA and various proteins involved in transcription, translation, or post-translational modifications. Non-limiting examples of protein-lipid interactions may include protein-hormone interactions, antibody-lipid interactions, interactions between a protein and a vesicle, the interaction of bovine β-lactoglobin and palmitate, and the interaction between intestinal fatty acid-binding protein and myristate. Non-limiting examples of protein-carbohydrate interactions may include the interaction of selectin and its ligand. Non-limiting examples of protein-small molecule interactions may include interactions between a protein and a pharmaceutical compound, a protein and an inhibitor of the protein, and a protein and an activator of the protein, where the pharmaceutical compound, inhibitor, or activator is a small molecule. There are many examples of protein-intermediate-protein interactions, because the intermediate may be any one of a number of different entities, including an ion, a co-repressor, a co-activator, or a scaffolding protein. Non-limiting examples of protein-microbe interactions may include protein-virus interactions, protein-bacterium interactions, protein-archaean interactions, protein-fungus interactions, and protein-protist interactions.

The interactions between biomolecules may be detected in vitro or in vivo. In vitro systems include systems for detecting biomolecule interactions in lysates or whole cells. Interactions between biomolecules may be detected by the detectable signal(s) generated by the associated heterologous polypeptide fragments. As detailed above, the optically detectable signals that may be generated include fluorescent, luminescent, phosphorescent, and colorimetric signals and. Such signals may be generated and quantified in living cells, thereby allowing for the real time characterization of the affinity, dynamics, and modulation of biomolecule-biomolecule interactions in biochemical pathways in living cells.

In some embodiments, interactions between two biomolecules may be detected. For this, the method comprises using an heterologous complementation system comprising a first and second nucleic acid construct, as detailed above. If the two biomolecules interact, the first and second heterologous polypeptide fragments are brought into close proximity and can thereby complement each other to form a functional protein capable of producing a detectable signal.

In another embodiments, interactions between more than two biomolecules may be detected. For example, interactions between three biomolecules may be detected. For this, the method comprises contacting a third nucleic acid construct with the first and second nucleic acid constructs (as defined above). The third nucleic acid construct has a nucleotide sequence encoding a third biomolecule and a nucleotide sequence encoding a third polypeptide fragment, wherein the third biomolecule has affinity for the second biomolecule. Furthermore, the first and third polypeptide fragments each comprise an N-terminal ends of a polypeptide and the second polypeptide fragment comprises a C-terminal end of a heterologous polypeptide. Thus, if the third biomolecule interacts with the second biomolecule, then a different detectable signal is produced upon association between the third polypeptide fragment and the second polypeptide fragment than upon interaction between the first and second biomolecules (see FIG. 11C). When the polypeptide fragments are derived from luciferase, the color of the signal is determined by the N-terminal polypeptide fragment. Alternatively, the heterologous complementation system may be constructed to encode one N-terminal polypeptide fragment and two C-terminal polypeptide fragments (each linked with an appropriate first, second, or third biomolecule). Thus, interaction between one set of biomolecules generates one detectable signal, and interaction between the other set of biomolecules generates a different signal.

In further embodiments, interactions between four or more biomolecules may be detected upon construction of the appropriate heterologous complementation systems, which comprise four or more nucleic acid constructs.

a. Survival-Selection Systems

In a preferred embodiment, the method of the invention may be employed in a survival-selection system. A variety of proteins suitable for the construction of a survival-selection system may be used as heterologous polypeptide fragments in the present invention. Survival-selection assays are typically based on dominant or recessive selection, including selection based on the conferment of drug resistance or metabolic selection. In accordance with this, examples of proteins that the first and second polypeptide may be independently selected from include aminoglycoside kinase (AK), beta-lactamase, thymidine kinase, hygromycin-B-phosphotransferase, adenosine deaminase, L-histidinol:NAD+ oxidoreductase, xanthine-guanine phosphoribosyl transferase (XPRT), glutamine synthetase, asparagine synthetase, puromycin N-acetyltransferase, aminoglycoside phosphotransferase, bleomycin binding protein, cytosine methyltransferase, O6-alkylguanine alkyltransferase, glycinamide ribonucleotide (GAR) transformylase, glycinamide ribonucleotide synthetase, phosphoribosyl-aminoimidazole synthetase, formylglycinamide ribotide amidotransferase, phosphoribosyl-aminoimidazole carboxamide formyltransferase, fatty acid synthease, IMP dehydrogenase, as well as any other protein that enables cell survival or growth under specific conditions.

These and similar proteins may be dissected into fragments, a first and a second polypeptide, corresponding to an N-terminal fragment and a C-terminal fragment and used in conjunction with the present invention, such that a cell survives under certain conditions if the first and the second polypeptide come together and form a functional protein. It will be apparent to one skilled in the art that a variety of measures of cell survival or cell growth may be employed for detection, including cell number, cell DNA content or protein content, cell size or shape, optical density, staining, and other measures.

b. Use of Constructs in Animal Models

Another aspect of the invention employs in vivo use of the nucleic acid construct pairs. In one embodiment, cells expressing one or both of the construct pairs may be injected or implanted into a model animal. The injected or implanted cells may be subsequently tracked using the detectable signal produced by the heterologous polypeptide fragments. The model animal may be selected from the group comprising mice, transgenic mice, rats, hamsters, fish, zebra fish, frogs, dogs, or primates. In another embodiment, cells expressing both construct pairs may be injected or implanted into a model animal and subsequently an intermediate may be administered to the model animal so as to induce association of the two constructs and produce a detectable signal from the heterologous polypeptide fragments.

In another embodiment, one or more nucleic acid constructs may be integrated into the genome of a model animal using techniques known in the art. In a further embodiment, one construct may be integrated into the genome of a model animal, and cells expressing the second construct may be injected or implanted into the same model animal. The injected or implanted cells may be subsequently tracked using the detectable signal produced by the heterologous polypeptide fragments. In yet another embodiment two or more nucleic acid constructs may be integrated into the genome of a model animal and the interaction of the biomolecules encoded by the construct pairs may be tracked using the detectable signal produced by the heterologous polypeptide fragments. In yet another embodiment two or more nucleic acid construct pairs may be integrated into the genome of a model animal and an intermediate may be administered to the model animal so as to induce association of the constructs and produce a detectable signal from the heterologous polypeptide fragments.

In another embodiment, a construct may be introduced into a model animal. The method of introduction may be oral, intra-peritoneal, intra-vascular, or intra-muscular. In another embodiment, one nucleic acid construct may be integrated into the genome of a model animal, and the second construct may be introduced into the model animal. In another embodiment, one nucleic acid construct may be integrated into the genome of a model animal, the second construct may be introduced into the model animal, and an intermediate may be administered to the model animal so as to induce association of the two constructs and produce a detectable signal from the heterologous polypeptide fragments. In the above embodiments, the nucleic acid construct that is integrated into the genome will be transcribed and translated to produce a complementation systems of the invention.

The construct pairs of the invention may be utilized in applications known in the art involving protein complementation. For example, the construct pairs may be used in any of the applications detailed in U.S. Patent Application Publication No. 2005/0144661, which is hereby incorporated by reference in its entirety.

c. Selection of Heterologous Polypeptide Fragment Pairs

Once the heterologous polypeptides are selected in accordance with the methods described herein, the preparation of heterologous polypeptide fragments, a first polypeptide fragment and a second polypeptide fragment, which may associate to produce one or more detectable signals, may be initiated by creating at least two distinct selection constructs - a N-terminus construct and a C-terminus construct. The N-terminus construct corresponds to the first polypeptide fragment, and the C-terminus construct corresponds to the second polypeptide fragment. The N-terminus construct comprises the nucleotide sequence encoding the N-terminal portion of a polypeptide, and the C-terminus construct comprises the nucleotide sequence encoding the C-terminal portion of a heterologous polypeptide. The selection constructs may then be incrementally truncated, as described below, and the resulting truncated constructs are then screened to identify pairs of truncated constructs that may associate to form a functional protein that produces a detectable signal.

The method of incremental truncation is summarized below, and additional details can be found in Ostermeier et al. (Proc. Natl. Acad. Sci. USA, 96:3562-3567,1999). Each selection construct is amplified and subsequently exposed to an exonuclease that unidirectionally digests the selection construct. For example, exonuclease III will remove nucleic acid bases from a 3′ recessed DNA end, but not a 5′ recessed end (Ostermeier et al., 1999). The selection constructs may be designed with an endonuclease restriction site in close proximity to the nucleotide sequence encoding the C-terminal portion of the N-terminus construct or N-terminal portion of the C-terminus construct. The action of the endonuclease thus leaves a 3′ recessed end at the C-terminal portion of the N-terminus construct and at the N-terminal portion of the C-terminus construct. Subsequent exposure of the constructs to a unidirectional exonuclease, such as exonuclease III, results in the removal of nucleic acids from the 3′ recessed DNA ends, created by the action of the endonuclease. The use of the endonuclease, prior to the use of the exonuclease, allows one to control the direction of digestion by the exonuclease. For the N-terminus construct, digestion should move towards the N-terminus of the polypeptide. For the C-terminus construct, digestion should move towards the C-terminus of the polypeptide.

Digestion by the exonuclease is allowed to proceed, and aliquots of digested selection constructs are removed at different time intervals and pooled. The resulting pool of constructs is comprised of nucleic acid constructs that have been incrementally truncated. The incrementally truncated constructs are then ligated into plasmids and transfected into cells. The cells are then examined for the desired detectable signal or signals, i.e., bioluminescence. Cells that display a detectable signal or signals are selected, and the selection constructs are rescued. The rescued constructs are then retransformed into cells to verify that construct pairs and not individual constructs generate the signal. After verification, the selection constructs are sequenced and the sequences are used to create nucleic acid constructs for use in the protein complementation assays of the invention. The examples illustrate use of the foregoing method to select suitable polypeptide fragments from a firefly luciferase.

Alternatively, once a first and second polypeptide fragment has been selected using the method described above, it is possible to select polypeptide fragments from other heterologous proteins by comparing secondary structure among the heterologous proteins. New polypeptide fragments typically share a similar secondary structure with the known polypeptide fragments (see FIG. 3 and the Examples).

### Definitions

As used herein, the term “biomolecule” refers to an organic molecule or a complex of organic molecules. Examples include a protein or fragments thereof, a nucleic acid, a carbohydrate, a lipid, a microbe, an organic small molecule, or a macromolecular complex. The macromolecular complex may contain a metal within a metal binding site.

As used herein, the term “carbohydrate” refers to a group of organic compounds that includes sugars, starches, celluloses, and gums. Carbohydrates may be monosaccharides, disaccharides, or polysaccharides.

As used herein, the term “detectable signal” refers to an optical signal, a radioisotopic radioactive emission signal, or a survival signal. An optical signal may be a signal detected through changes in light emission, such as a fluorescence, luminescence, bioluminescence, phosphorescence, or color. A radioactive emission signal is represented by isotopic decay and measuring radioactivity emitted from biological material, cells or animals. A survival signal is represented by the growth of an otherwise compromised cell.

As used herein, the term “heterologous polypeptide fragments” refers to two polypeptide fragments wherein the first polypeptide fragment and the second polypeptide fragment are 1) derived from the same protein family but from different members within the family, 2) derived from the same protein family but from different organisms or 3) derived from different protein families but from the same organism.

As used herein, the term “lipid” refers to a group of organic compounds that includes fats, oils, waxes, sterols, and triglycerides. Lipids are typically water insoluble. Examples include fatty acids, triacylglycerols, fatty-acid esters, sphingoids, glycolipids, phospholipids, sphingolipids, carotenes, polyprenols, sterols, terpenes, or isoprenoids.

The term, “overlap region,” as used herein, refers to a region of structural similarity or sequence similarity between the first polypeptide fragment and the second polypeptide fragment.

The term “small molecule” refers to a compound, which has a molecular weight of less than about 5 kD, less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids or other organic (carbon containing) or inorganic molecules.

As used here, the term “selection construct” refers to a nucleic acid construct that is comprised of either the N-terminus or the C-terminus of a protein that is capable of producing a detectable signal, and at least one restriction endonuclease site.

As various changes could be made in the above-described methods and compositions without departing from the scope of the invention, it is intended that all matter contained in the above description and the examples presented below, shall be interpreted as illustrative and not in a limiting sense.

### EXAMPLES

### Example 1

### Initial Library Construction and Screening

The initial library was constructed as described in Luker et. al., 2004 (supra), and described briefly below. This library employed a well-characterized protein interaction system: rapamycin-mediated association of the FRB domain of human mTOR (residues 2024-2113) with FKBP-12. The Ser-Thr kinase mTOR is inhibited by FKBP-12 in a rapamycin-dependent manner. The inventors chose FRB, the 11 kD domain of human mTOR that binds with high affinity to the rapamycin-FKBP complex, to construct and screen a comprehensive incremental truncation library for enhanced LCI (luciferase complementation imaging). Initial N-terminal and C-terminal fragments of firefly (Photinus pyralis) luciferase were amplified from pGL3-Control (Promega, Madison, Wis.). The FRB domain of human mTOR (residues 2024-2113) and human FKBP-12 (Promega) were generated by PCR amplification from plasmids kindly provided by X. F. Zheng (Washington University in St. Louis). A flexible Gly/Ser linker and a multiple cloning site (BgIII, BsiWI, MIuI, SmaI) were added using synthetic oligonucleotide primers. The fusions were expressed in E. coli in plasmids pDIM-N2 and pDIM-C6 (gift of S. Benkovic, Pennsylvania State University) (Ostermeier et al., 1999, supra) and isolated.

Briefly, N- and C-terminal incremental truncation libraries were constructed by unidirectional digestion with exonuclease III (Exo III) essentially as described (Ostermeier et al., 1999). Both libraries were characterized by sequencing and restriction digest of randomly chosen clones to confirm that the obtained truncations covered the full length of each luciferase fragment. Libraries were packaged in phage and E. coli were co-infected with phage libraries, followed by selection on LB agar plates containing 50 μg/ml ampicillin, 50 μg/ml chloramphenicol, 0.3 mM IPTG and 1 μM rapamycin. To visualize positive clones, colonies were blotted to sterile nitrocellulose filters. Filters were moistened with substrate solution (1 mM D-luciferin in 0.1 M sodium acetate (pH 5.0) for 3-5 min), and then imaged with an IVIS CCD camera (Xenogen, Alameda, Calif.; 1 min exposure, f-stop=1, binning 8, FOV 15 cm) to locate bioluminescent colonies. Clones of interest were isolated and re-tested for rapamycin-inducible bioluminescence.

Plasmids were rescued from these clones, separated, and re-transformed into E. coli to confirm that plasmid pairs, not single plasmids, recapitulated the original phenotype. The extent of deletion in the optimal LCI pair was characterized by sequencing. The optimal pair is depicted in FIG. 2. Fusions were amplified with primers adding a Kozak consensus sequence to the 5′ end and ligated into mammalian expression vectors pcDNA3.1 TOPO (FRB-NLuc) and pEF6-TOPO (CLuc-FKBP) (Invitrogen, Carlsbad, Calif.).

### Example 2

### Designing a Heterologous Complementation System

To improve the system for use in vivo, heterologous luciferase proteins were used to create polypeptide fragments with two essential properties. First and foremost, the pair needed to function well. Also, for selected applications, the detectable signal needed to be inducible. Suitable inducibility requires a low residual activity of the pairs when not associated and a high degree of activity upon induced approximation of the pair. The level of inducibility can suffer from either too high a background signal or too low a final output.

There are several variants of luciferase proteins (Photinus pyralis, Pyrophorus plagiophthalmus) that utilize the same substrate (D-luciferin). These proteins however, exhibit vastly different amino acid sequences and emission characteristics. The spectral output can vary from 530 nm (blue/green) up into the far-red range (630 nm) and exhibit different kinetics. Although firefly luciferase and click beetle luciferases (Pyrophorus plagiophthalmus) only retain about 48% amino acid homology, secondary structure predictions revealed that they were quite similar (see FIG. 3). Click beetle luciferases exhibit a large separation in spectral emission with click beetle red (CBR) emitting at λmax=615 nm and click beetle green (CBG; CBG68 and CBG99) emitting at λmax=540 nm. Click beetle green and click beetle red luciferases exhibit 98.5% amino acid homology. Firefly luciferase and the family of click beetle luciferases are heterologous proteins that share only modest sequence homology, but share high domain homology thereby fostering the possibility of creating polypeptide fragment pairs with varying spectral emissions, while utilizing the same substrate. Each heterologous protein was split such that a previously identified critical overlap region was retained on the N-terminal and C-terminal fragments (see FIG. 3). This overlap region spans residues 397-416 of firefly luciferase (MSGYVNNPEATNALIDKDG) (SEQ ID NO:1) (Luker et al., 2004) and residues 395-413 of CBR and CBG (SKGYVNNVEATKEAIDDDG) (SEQ ID NO:2), resulting in a 64% sequence homology for the overlap sequence. Therefore, the click beetle constructs comprised residues 2-413 (i.e., N-terminal portion) and 395-542 (i.e., the C-terminal portion) (see FIG. 4). PCR was used to introduce convenient correct restriction sites, into the click beetle luciferases for cloning, as well as flexible G-S linkers and/or translational control sites. The PCR primers are presented in Table 1 and the PCR strategy is outlined in FIG. 5.

TABLE 1
PCR Primers.

| | | | |
|---|---|---|---|
| | | | SEQ |
| Primer | Sequence | Length | ID NO: |

| | | | |
|---|---|---|---|
| N-term- | CGTACGCGTCCCGGGGCGGTGGCTCAT | 73 | 3 |
| CBR- | CTGGCGGAGGTGTAAAGCGTGAGAAAA | | |
| CBG99-fwd | ATGTCATCTATGGCCCTGA | | |
| N-term- | CTCGAGTTAGCCGTCGTCGTCGATGGC | 37 | 4 |
| CBR-rev | CTCCTTGGTA | | |
| C-term- | GGATCCACTAGTCCAGTGTGGTGGAAT | 88 | 5 |
| CBR-fwd | TGCCCTTACACCACCGCCACCATGAAG | | |
| | GGTTATGTCAATAACGTTGAAGCTACC | | |
| | AAGGAGG | | |
| C-term- | CGTACGAGATCTGACCTCCGCCAGATG | 64 | 6 |
| CBR-rev | AGCCACCTCCACCGCCGGCCTTCACCA | | |
| | ACAATTGTTT | | |
| C-term- | CGTACGAGATCTGACCTCCGCCAGATG | 64 | 7 |
| CBG-rev | AGCCACCTCCACCGCCGGCCTTCTCCA | | |
| | ACAATTGTTT | | |
| N-term- | CGTACGCGTCCCGGGGCGGTGGCTCAT | 65 | 8 |
| CBG68-fwd | CTGGCGGAGGTATGGAGATGTGGCATG | | |
| | AAGGCCTGGAA | | |
| N-term- | CGTACGCGTCCCGGGGCGGTGGCTCAT | 73 | 9 |
| CBR/CBG | CTGGCGGAGGTGTAAAGCGTGAGAAAA | | |
| 99-fwd | ATGTCATCTATGGCCCTGA | | |
| N-term- | CTCGAGTTAGCCGTCGTCGTCGATGGC | 37 | 10 |
| CBR-rev | CTCCTTGATT | | |
| C-term- | GGATCCACTAGTCCAGTGTGGTGGAAT | 89 | 11 |
| CBR-fwd | TGCCCTTACACCACCGCCACCATGGAA | | |
| | GGGTTATGTCAATAACGTTGAAGCTAC | | |
| | CAAGGAGG | | |
| C-term- | CGTACGAGATCTGACCTCCGCCAGATG | 64 | 12 |
| CBR-rev | AGCCACCTCCACCGCCGGCCTTCACCA | | |
| | ACAATTGTTT | | |

The PCR products were ligated into a TOPO sequencing vector (Invitrogen) and then transferred into the original Firefly luciferase vectors, as described in Example 1 (Luker et al., 2004). All the vectors were then transferred into the TriEx3Neo triple expression vector (Novagen, Madison, Wis.) to assure equivalent expression levels in transient transfection.

### Example 3

### Measuring Luminescence

Combinations of several possible polypeptide fragments (F=firefly luciferase, R=red click beetle luciferase, G=green click beetle 99 luciferase, N=N-terminus construct with FRP, C=C-terminus construct with FKBP) were assayed using rapamycin-inducible interaction of FRB with FKBP fused to the N-terminal and C-terminal fragments, respectively. (NOTE—CBG99 and CBG68 produce essentially identical constructs and thus, for illustration purposes, only the CBG99 data will be shown.) (FIGS. 6-14) The constructs were transfected into HEK293T cells using Fugene6 (Roche Applied Sciences, Indianapolis, Ind.) in a ratio of 3 μL for every μg of DNA. The experiments were set up in a 96 well plate format. Each well was transfected with 75 ng DNA, 225 nL Fugene6, and 10,000 cells in DMEM and heat-inactivated FBS. The cells were allowed to incubate for 48 hours. Rapamycin at a final concentration of 100 nM was added, and the cells subsequently incubated for 8 hours. After 8 hours, images were taken using the Xenogen IVIS 100 optical imager. The cells were imaged in MEBSS supplemented with 1% serum and 150 μg/mL of D-luciferin. The plates were incubated at 37° C. for 10 minutes and then imaged. One minute images were taken at all available filters with a FOV C and binning of 4. Experimental DNA was prepared in molar equivalent ratios to ensure equal parts intact protein and recombined heteroprotein fragments of the various split luciferases. Renilla luciferase was also transfected to use as a transfection control as described by Pichler et al. 2005, supra.

Hetero-polypeptide fragment combinations worked for all tested pairs except firefly N with any click beetle C (FIG. 6A). Photon output for GN+GC, GN+RC, RN+GC, and RN+RC exceeded the original split firefly luciferase complementation pair. In particular, the photon output for GN+GC exceeded the original split firefly luciferase complementation pair by 5-fold. Also the pair exhibiting the highest output retained approximately 20% of the total flux of the intact firefly luciferase. Most reconstituted enzyme pairs displayed only 1% or less of the enzyme activity of the original intact protein. FIG. 6B is presented in log scale and better illustrates that combinations of either GN or RN with FC yielded low, but significant photon flux (˜4 fold over background). Additionally, while some pairs displayed better overall intensity, others also demonstrated a higher rapamycin-dependent fold-induction than the original split luciferase pair. For example, GN+GC showed 75-fold rapamycin-dependent induction, 3-5 fold greater than the original split Firefly luciferase. Furthermore, the interaction was specific (FIG. 7). A point mutation in FRB (S2035I) known to abrogate rapamycin-induced FRB-FKBP binding completely blocked rapamycin-induced protein association.

Quantitative output could be generated by the novel hetero-protein polypeptide fragment pairs (FIG. 8). For example, using all productive click beetle green or red fragments in live HEK293T cells, a K_{d} of 0.25 +/−0.05 nM for rapamycin-dependent induction of FRB and FKBP binding was determined, nearly identical to literature values. Similarly, a Kᵢ of 1.57 +/−0.86 nM was determined for FK506 inhibition of rapamycin-induced FRB and FKBP binding. Overall, GN+GC showed the highest fold-induction combined with the highest total photon flux (FIGS. 9 and 10). It exceeded split firefly on both properties. As might be anticipated, the emission spectra of all the pairs tested varied. It was discovered that the N-terminus fragment dictated the color output. For example, HEK293T cells were transfected for 48 hours with various combinations of the complementation fragments and then incubated for 8 hours with 100 nM rapamycin. Red and green emissions were detected with a 590 nm long-pass filter and 500-570 nm band-pass filter, respectively. Relative color was determined from the 60 sec images in each filter and calculating the ratio of red emission to green emission. GN+GC, GN+RC and GN+FC all showed a high green output (low red/green ratio), while RN+GC, RN+RC and RN+FC all showed a high red color output (high red/green ratio) (FIGS. 10 and 11). This unexpected property can be exploited in combination with spectral unmixing to enable novel applications in systems biology as shown below.

To illustrate spectral unmixing applications, various ratios of DNA from the indicated split pairs were transfected into HEK293T cells and imaged with red (>590 nm) and green (500-570 nm) filters to separate the colors as shown in the lower two panels. Using a previously published bioluminescence unmixing algorithm (Gammon et al. 2006, supra), it was then possible to quantify different luciferase colored-tagged protein interactions simultaneously. Thus, the location and type of various colored luciferase-tagged protein interactions (e.g., FN+FC and GN+GC) could be identified on the 96-well plates. In another preferred implementation, varying DNA concentrations were co-transfected into the same cells. Herein, two protein interaction pairs could be simultaneously imaged and deconvoluted in the same cells (e.g., FN+FC and GN+GC) with two different colored filters (FIG. 12A) or importantly, two different N-terminal tagged proteins could be simultaneously imaged and deconvoluted (e.g., RN+GN+GC) in the same cells (FIG. 12B). The latter example provides a novel tool for interrogation of branch points in protein-protein interaction pathways and molecular switches in living cells in real time.

When imaging in vivo, background signal and light penetration limits the dynamic range of optical probes for detecting protein interactions. In the case of luciferase complementation assays, background consists of bioluminescence in animals and cells, which in cells is extremely low, but spontaneous bioluminescence in animals can be somewhat higher, especially in the gut. To limit these problems in imaging, the output of the reporter should be made as high as possible and the color should be as red as possible. Representative mice imaged pre and post rapamycin illustrate the use of several polypeptide fragment pairs in live animals (FIGS. 13-14). Hepatocellular somatic gene transfer was performed by hydrodynamic injections of 15 mg total DNA per mouse. After 18 hours, mice were injected with 150 mg/kg D-Luciferin IP and imaged with an IVIS 100 CCD camera to establish baseline photon output. Mice were then treated with rapamycin (IP: 1 mg/kg) and re-imaged 6 hours post-rapamycin to visualize induced protein complementation in vivo (FIG. 13). Coelenterazine (IV: 1 mg/kg) was also injected as described to measure Renilla luciferase activity for monitoring transfection efficiency. D-Luciferin photon output (split luciferases) was normalized to coelenterazine photon output (Renilla luciferase) and is presented as a ratio of Split Luc/Renilla (FIG. 14). Inset (FIG. 14B) shows the average +/− SEM of all mouse data.

Herein, several pairs, including RN+GC and GN+GC, exhibit a favorable combination of color, output, and fold-induction, yielding enhanced heterologous polypeptide fragment pairs for imaging biomolecule interactions in vivo.

What is claimed is:

1. A heterologous complementation system comprising a first nucleic acid construct and a second nucleic acid construct, the first nucleic acid construct having a nucleotide sequence encoding a first biomolecule and a nucleotide sequence encoding a first polypeptide fragment, the second nucleic acid construct having a nucleotide sequence encoding a second biomolecule and a nucleotide sequence encoding a second polypeptide fragment, the first and second polypeptide fragments each being derived from a heterologous polypeptide, wherein when the first biomolecule, the first polypeptide fragment, the second biomolecule, and the second polypeptide fragment are each expressed, and if the first biomolecule interacts with the second biomolecule, then the first polypeptide fragment associates with the second polypeptide fragment to produce a detectable signal.

2. The heterologous complementation system of claim 1, wherein the first and second biomolecules are independently selected from the group consisting of a protein, a peptide, an enzyme, a receptor, an antibody, a small molecule, a ligand, an antigen, a nucleic acid, a lipid, a carbohydrate, and a microbe.

3. The heterologous complementation system of claim 1, wherein the first and second polypeptide fragments are selected from the group consisting of enzyme fragments, beta-galactosidase fragments, beta-lactamase fragments, dihydrofolate reductase fragments, luciferase fragments, and fluorescent protein fragments.

4. The heterologous complementation system of claim 1, wherein the first polypeptide fragment is an N-terminal region of a first luciferase, and the second polypeptide fragment is a C-terminal region of a second luciferase.

5. The heterologous complementation system of claim 4, wherein the first and second luciferases are independently selected from the group consisting of click beetle red luciferase, click beetle green luciferase, and firefly luciferase.

6. The heterologous complementation system of claim 5, wherein the C-terminal end of the first luciferase overlaps with the N-terminal end of the second luciferase.

7. The heterologous complementation system of claim 6, wherein the region of overlap comprises amino acid residues 395 to 416 of each luciferase.

8. The heterologous complementation system of claim 1, wherein the detectable signal is an optical signal selected from the group consisting of fluorescence, luminescence, phosphorescence, and a colorimetric signal.

9. The heterologous complementation system of claim 1, wherein the first nucleic acid construct and the second nucleic acid construct comprise:
R1-R2-R3 and
R6-R7-R8, respectively;
wherein:
R1 is a nucleotide sequence encoding the first biomolecule;
R2 is a nucleotide sequence encoding a flexible linker;
R3 is a nucleotide sequence encoding the first polypeptide fragment;
R6 is a nucleotide sequence encoding the second polypeptide fragment;
R7 is a nucleotide sequence encoding a flexible linker; and
R8 is a nucleotide sequence encoding the second biomolecule.

10. The heterologous complementation system of claim 9, wherein the first biomolecule is FRP; the first polypeptide fragment is an N-terminal region of a first luciferase; the second polypeptide fragment is a C-terminal region of a second luciferase; the second biomolecule is FKBP; and each linker comprises glycine, serine, or a combination thereof.

11. The heterologous complementation system of claim 10, wherein the first luciferase is click beetle green luciferase and the second luciferase is click beetle red luciferase.

12. The heterologous complementation system of claim 10, wherein the first luciferase is click beetle red luciferase and the second luciferase is click beetle green luciferase.

13. The heterologous complementation system of claim 1, wherein the first and second nucleic acid constructs are linked and comprise one molecule.

14. A method for detecting molecular interactions, the method comprising:
a. combining a first nucleic acid construct having a nucleotide sequence encoding a first biomolecule and a nucleotide sequence encoding a first polypeptide fragment with a second nucleic acid construct having a nucleotide sequence encoding a second biomolecule and a nucleotide sequence encoding a second polypeptide fragment under conditions such that the first biomolecule, the first polypeptide fragment, the second biomolecule and the second polypeptide fragment are expressed, the first polypeptide fragment and the second polypeptide fragment each being derived from a heterologous polypeptide; and
b. determining whether a detectable signal is produced, the detectable signal being produced by association between the first polypeptide fragment and the second polypeptide fragment if the first biomolecule interacts with the second biomolecule.

15. The method of claim 14, wherein the first and second biomolecules are independently selected from the group consisting of a protein, a peptide, an enzyme, a receptor, an antibody, a small molecule, a ligand, an antigen, a nucleic acid, a lipid, a carbohydrate, and a microbe.

16. The method of claim 14, wherein the first and second polypeptide fragments are selected from the group consisting of enzyme fragments, beta-galactosidase fragments, beta-lactamase fragments, dihydrofolate reductase fragments, luciferase fragments, and fluorescent protein fragments.

17. The method of claim 14, wherein the first polypeptide fragment is an N-terminal region of a first luciferase, and the second polypeptide fragment is a C-terminal region of a second luciferase.

18. The method of claim 17, wherein the first and second luciferases are independently selected from the group consisting of click beetle red luciferase, click beetle green luciferase, and firefly luciferase.

19. The method of claim 18, wherein the C-terminal end of the first luciferase overlaps with the N-terminal end of the second luciferase.

20. The method of claim 19, wherein the region of overlap comprises amino acid residues 395 to 416 of each luciferase.

21. The method of claim 14, wherein the detectable signal is an optical signal selected from the group consisting of fluorescence, luminescence, phosphorescence, and a colorimetric signal.

22. The method of claim 14, wherein the first nucleic acid construct and the second nucleic acid construct comprise:
R1-R2-R3 and
R6-R7-R8, respectively;
wherein:
R1 is a nucleotide sequence encoding the first biomolecule;
R2 is a nucleotide sequence encoding a flexible linker;
R3 is a nucleotide sequence encoding the first polypeptide fragment;
R6 is a nucleotide sequence encoding the second polypeptide fragment;
R7 is a nucleotide sequence encoding a flexible linker; and
R8 is a nucleotide sequence encoding the second biomolecule.

23. The method of claim 22, wherein the first biomolecule is FRP; the first polypeptide fragment is an N-terminal region of a first luciferase; the second polypeptide fragment is a C-terminal region of a second luciferase; the second biomolecule is FKBP; and each linker comprises glycine, serine, or a combination thereof.

24. The method of claim 23, wherein the first luciferase is click beetle green luciferase and the second luciferase is click beetle red luciferase.

25. The method of claim 23, wherein the first luciferase is click beetle red luciferase and the second luciferase is click beetle green luciferase.

26. The method of claim 14, wherein the first and second nucleic acid constructs are linked and comprise one molecule.

27. The method of claim 14, further comprising combining the first and second nucleic acid constructs with a third nucleic acid construct having a nucleotide sequence encoding a third biomolecule and a nucleotide sequence encoding a third polypeptide fragment, the third biomolecule having affinity only for the second biomolecule, wherein a different detectable signal is produced by association between the third polypeptide fragment and the second polypeptide fragment if the third biomolecule interacts with the second biomolecule.


SEQUENCE LISTING (12 sequences)

SEQ ID NO: 1 - protein, 19 aa, Lampyridae
MSGYVNNPEATNALIDKDG

SEQ ID NO: 2 - protein, 19 aa, Chalcolepidius
SKGYVNNVEATKEAIDDDG

SEQ ID NO: 3 - DNA, 73 bp, Chalcolepidius
cgtacgcgtcccggggcggtggctcatctggcggaggtgtaaagcgtgagaaaaatgtcatctatggccctga

SEQ ID NO: 4 - DNA, 37 bp, Chalcolepidius
ctcgagttagccgtcgtcgtcgatggcctccttggta

SEQ ID NO: 5 - DNA, 88 bp, Chalcolepidius
ggatccactagtccagtgtggtggaattgcccttacaccaccgccaccatgaagggttatgtcaataacgttgaagctaccaaggagg

SEQ ID NO: 6 - DNA, 64 bp, Chalcolepidius
cgtacgagatctgacctccgccagatgagccacctccaccgccggccttcaccaacaattgttt

SEQ ID NO: 7 - DNA, 64 bp, Chalcolepidius
cgtacgagatctgacctccgccagatgagccacctccaccgccggccttctccaacaattgttt

SEQ ID NO: 8 - DNA, 65 bp, Chalcolepidius
cgtacgcgtcccggggcggtggctcatctggcggaggtatggagatgtggcatgaaggcctggaa

SEQ ID NO: 9 - DNA, 73 bp, Chalcolepidius; identical to SEQ ID NO: 3

SEQ ID NO: 10 - DNA, 37 bp, Chalcolepidius
ctcgagttagccgtcgtcgtcgatggcctccttgatt

SEQ ID NO: 11 - DNA, 89 bp, Chalcolepidius
ggatccactagtccagtgtggtggaattgcccttacaccaccgccaccatggaagggttatgtcaataacgttgaagctaccaaggagg

SEQ ID NO: 12 - DNA, 64 bp, Chalcolepidius; identical to SEQ ID NO: 6